(19) United States
(12) Patent Application Publication
(10) Pub. No.: US 2023/0167121 A1
(43) Pub. Date: Jun. 1, 2023

(54) SUBSTITUTED {1,2,4,} TRIAZOLO{1,5-A} PYRIMIDINE COMPOUNDS AND USE IN STABILIZING MICROTUBULES

(71) Applicants: THE TRUSTEES OF THE UNIVERSITY OF PENNSYLVANIA, Philadelphia, PA (US); THE REGENTS OF THE UNIVERSITY OF CALIFORNIA, Oakland, CA (US)

(72) Inventors: Kurt R. BRUNDEN, Media, PA (US); John Q. TROJANOWSKI, Philadelphia, PA (US); Amos B. SMITH, III, Merion, PA (US); Virginia M-Y LEE, Philadelphia, PA (US); Carlo BALLATORE, San Diego, CA (US); Thibault ALLE, La Jolla, CA (US)

(21) Appl. No.: 17/995,761

(22) PCT Filed: Apr. 12, 2021
(86) PCT No.: PCT/US2021/026791

§ 371 Date: Oct. 7, 2022

Related U.S. Application Data
(60) Provisional application No. 63/009,727, filed on Apr. 14, 2020.

(51) Int. Cl. C07D 487/04

(52) U.S. Cl. CPC C07D 487/04

(57) ABSTRACT

Provided are nitrile- and alkyne-substituted {1,2,4,} triazolo {1,5-A} pyrimidine compounds, compositions containing such compounds, and methods for treating a neurodegenerative disease or cancer by administration of such compounds.

### CROSS-REFERENCE TO RELATED APPLICATIONS

The present application claims the benefit of priority to U.S. Provisional Application No. 63/009,727, filed Apr. 14, 2020, the entire contents of which are incorporated herein by reference.

### GOVERNMENT RIGHTS

This invention was made with government support under AG044332 and AG061173 awarded by the National Institutes of Health. The government has certain rights in the invention.

### TECHNICAL FIELD

The invention is directed to compounds and methods for the treatment of cancer or neurodegenerative tauopathies such as Alzheimer's disease and frontotemporal lobar degeneration.

### BACKGROUND

Neurodegenerative tauopathies, including Alzheimer's disease (AD), are characterized by the misfolding and aggregation of the microtubule (MT)-associated protein tau. Normally, tau binds to and stabilizes MTs, thereby maintaining the network of MTs essential for axonal transport in neurons. In AD, tau becomes sequestered into aggregates, known as neurofibrillary tangles (NFTs) and neuropil threads, resulting in reduced MT-binding. This loss of tau function and/or the formation of oligomeric or fibrillar tau species is believed to lead to MT destabilization and consequent axonal transport deficits, which could result in neuronal dysfunction and death.

Other neurodegenerative diseases where MT function may be compromised include frontotemporal lobar degeneration, multiple sclerosis, Parkinson's disease, amyotrophic laterial sclerosis, schizophrenia, Huntington's disease, multiple sclerosis, and traumatic brain injury (TBI), especially repetitive TBI (rTBI) such as that due to dementia pugilistica and recurrent football concussions and military closed head injuries, which also is known as chronic traumatic encephalopathy (CTE).

Compounds that can cross the blood brain barrier and effectively stabilize MT are needed in order to treat neurodegenerative diseases caused, at least in part, by misfolding and aggregation of the MT-associated protein tau.

Originally reported as anti-fungal agents, microtubule (MT)-active [1,2,4]triazolo[1,5-a]pyrimidines and related heterocyclic molecules have since attracted attention as potential candidates for a variety of applications including cancer chemotherapy and neurodegenerative disease treatment. Cevipabulin (Compound 1) is a potent anti-cancer compound. This compound, like vincristine, can interact with tubulin heterodimers and interfere with the rate of exchange of the guanosine triphosphate (GTP) and, thus, competes with vincristine but not taxol or colchicine, for binding to MTs. See, Zhang, “Synthesis and SAR of [1,2,4]triazolo[1,5-a]pyrimidines, a class of anticancer agents with a unique mechanism of tubulin inhibition,” J. Med. Chem., 2007, 50:319-27.

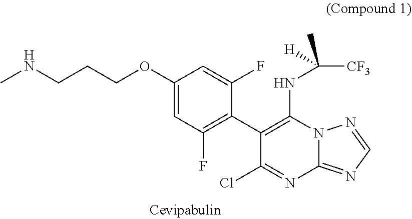 [SMILES: CNCCCOc1cc(F)c(-c2c(Cl)nc3ncnn3c2N[C@@H](C)C(F)(F)F)c(F)c1]

Opposite to the activity of vincristine/vinblastine, triazolopyrimidine Compound 2, binds exclusively to MTs and not to unpolymerized tubulin heterodimers. See Sáez-Calvo, “Triazolopyrimidines Are Microtubule-Stabilizing Agents that Bind the Vinca Inhibitor Site of Tubulin,” Cell Chemical Biology, 2017, 24, 737-750 e6.

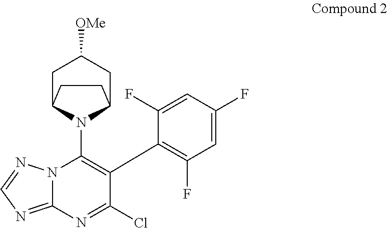 [SMILES: CO[C@H]1C[C@H]2CC[C@@H](C1)N2c1c(-c2c(F)cc(F)cc2F)c(Cl)nc2ncnn12]

Triazolopyrimidine structural modifications can promote MT stabilization or disrupt MT integrity. These differences can have important and unknown ramifications in the therapeutic applications of triazolopyrimidines, including exhibiting different binding modes.

There remains a need for appropriately substituted triazolopyrimidines compounds for treating neurodegenerative disease.

### SUMMARY

Provided are substituted {1,2,4,} triazolo{1,5-A} pyrimidine compounds according to Formula I:

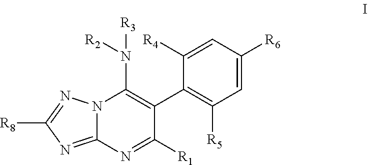

wherein

R₁ is Cl;

R₂ is CH(CH₃)CF₃, CH(isopropyl)CF₃, CH(isopropyl)CH₃, CH(tert-butyl)CH₃, or CH(methyl diazirinyl)CH₃, wherein R₂ may optionally be mono- or poly-deuterated;

R₃ is H or alkyl; or, R₂ and R₃, together with the N atom to which they are attached, form a C₅-C₇ heterocyclic ring or 3-methoxy-8-azabicyclo[3.2.1]octan-8-yl;

R₄ is H or F;

R₅ is H or F;

R₆ is iodo, cyano, ethynyl, or —C≡C—(CH₂)ₙ—R₇, wherein n=0-3, and R₇ is H, —OH, —NH₂, —NHCH₃, or —N(CH₃)₂, wherein R₆ may optionally be mono- or poly-deuterated; R₈ is H or alkyl;

or a stereochemical isomer thereof,

or a pharmaceutically acceptable salt thereof.

Also disclosed are compositions for treating a neurodegenerative disease or cancer comprising a therapeutically effective amount of a compound according to Formula I, and methods of treating a neurodegenerative disease or cancer in a subject in need thereof comprising administering to the subject a therapeutically effective amount of a compound according to Formula I.

### DETAILED DESCRIPTION

Microtubule (MT)-active [1,2,4] triazolo[1,5-a] pyrimidines (TPDs) and related heterocyclic molecules have attracted attention as potential candidates for applications in treatment of neurodegenerative disease. The present inventors have previously characterized a number of MT-stabilizing and MT-destabilizing TPDs. For example, U.S. Pat. No. 9,649,317 discloses methods of use for such compounds. The present disclosure provides novel TPDs containing specified C6-phenyl substitutions. Prior investigation into the MT stabilizing properties of TPD's in cell-based assays of MT-stabilization revealed that varying substitution patterns result in molecules that can either promote MT stabilization or conversely disrupt MT stabilization. Based on the observation that specific structural features within the TPD's are critical to elicit an increase in MT stability, the inventors undertook a more systemic exploration of the SAR of the TPD scaffold. The SAR work led to the synthesis of novel C6 nitrile and alkyne substituted TPDs, as described herein. Importantly, the presently disclosed molecules have improved MT-stabilizing activity and pharmaceutical properties relative to previously described examples.

Thus, it has now been discovered that certain classes of microtubule-stabilizing compounds will be useful in treating neurodegenerative diseases, in particular, tauopathies, for example, Alzheimer's disease, frontotemporal lobar degeneration, Pick's disease, progressive supranuclear palsy (PSP), and corticobasal degeneration. In addition, the compounds of the invention may be useful for other diseases where tau pathology is a co-morbidity or where microtubule function is compromised, for example, schizophrenia, Parkinson's disease (PD), PD with dementia, Lewy body disease with dementia, and amyotrophic lateral sclerosis.

The compounds of the invention can also be used to treat traumatic brain injury (TBI), especially repetitive TBI (rTBI), such as that due to dementia pugilistica and recurrent football concussions and military closed head injuries such as that due to IEDs, which also is known as chronic traumatic encephalopathy (CTE), with features of tauopathy or AD-like pathology. It is speculated that CTE also may emerge from PTSD.

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, “alkyl” refers to an optionally substituted, saturated straight, or branched, hydrocarbon radical having from about 1 to about 20 carbon atoms (and all combinations and subcombinations of ranges and specific numbers of carbon atoms therein). For example, “alkyl” may refer to a substituted or unsubstituted, straight or branched hydrocarbon radical having about 1-20, 1-15, 1-10, 1-8, 1-6, 1-5, 1-4, or 1-3 carbon atoms. In some embodiments, “alkyl” refers to an unsubstituted, straight radical having about 1-6 carbon atoms. Where appropriate, “alkyl” can mean “alkylene”; for example, if X is —R₁R₂, and R₁ is said to be “alkyl”, then “alkyl” may correctly be interpreted to mean “alkylene”.

“Heterocyclyl” or “heterocyclic” refers to a stable 3- to 18-membered non-aromatic ring radical that comprises two to twelve carbon atoms and from one to six heteroatoms selected from nitrogen, oxygen and sulfur. In some embodiments, the only heteroatom is the nitrogen atom to which R₂ and R₃ are attached. Whenever it appears herein, a numerical range such as “3 to 18” refers to each integer in the given range, e.g., “3 to 18 ring atoms” means that the heterocyclyl group may consist of 3 ring atoms, 4 ring atoms, et cetera, up to and including 18 ring atoms. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of heterocyclyl radicals include, but are not limited to, azepanyl, azocanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. “Heterocyclyl” may also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic. A heterocyclyl moiety is optionally substituted with one, two, or three substituents selected from halo (F, Cl, Br, or I, preferably F), —OH, —OC₁₋₆alkyl, —CN, —NH₂, —NH(C₁₋₆alkyl), —NH(C₁₋₆alkyl)₂, C₃₋₈cycloalkyl, heterocyclyl, aryl, or heteroaryl.

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

The present compounds include compounds of Formula I:

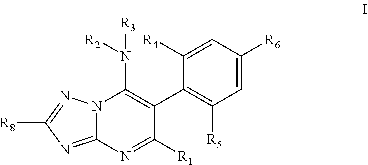

wherein

R₁ is Cl;

R₂ is CH(CH₃)CF₃, CH(isopropyl)CF₃, CH(isopropyl)CH₃, CH(tert-butyl)CH₃, or CH(methyl diazirinyl)CH₃, wherein R₂ may optionally be mono- or poly-deuterated;

R₃ is H or alkyl; or, R₂ and R₃, together with the N atom to which they are attached, form a C₅-C₇ heterocyclic ring or 3-methoxy-8-azabicyclo[3.2.1]octan-8-yl;

R₄ is H or F;

R₅ is H or F;

R₆ is iodo, cyano, ethynyl, or —C≡C—(CH₂)ₙ—R₇, wherein n=0-3, and R₇ is H, —OH, —NH₂, —NHCH₃, or —N(CH₃)₂, wherein R₆ may optionally be mono- or poly-deuterated;

R₈ is H or alkyl;

or a stereochemical isomer thereof,

or a pharmaceutically acceptable salt thereof.

Stereoisomeric forms of the compounds of Formula I, for example, enantiomers, diastereomers, and atropisomers, are also within the scope of the invention, as are pharmaceutically acceptable salts of any compound or stereoisomer of Formula I. As used herein, “stereoisomers” refers to all enantiomerically/diastereomerically pure and enantiomerically/diastereomerically enriched compounds of the invention. Atropisomers, that is, stereoisomers resulting from hindered rotation about single bonds, are also within the scope of the term, “stereoisomers.”

As used herein, “pharmaceutically acceptable salt” refers to a salt of a compound of the invention that is generally considered safe for pharmaceutical use. Examples include, for example, hydrochloric acid, sulfuric, fumaric, succinic, ascorbic, maleic, methanesulfonic, and isoethonic acid salts.

R₃ is H or alkyl. In certain embodiments, R₃ is H. In other embodiments R₃ is alkyl, such as C₁-C₆ alkyl.

In certain embodiments of the present compounds, R₆ is iodo. In some of these embodiments, R₄ and R₅ are both F, and R₃ is H. In the embodiments in which R₄ and R₅ are both F, R₂ may be, for example, CH(CH₃)CF₃, CH(isopropyl)CF₃, CH(isopropyl)CH₃, or CH(tert-butyl)CH₃.

In certain embodiments of the present compounds, R₆ is cyano. In some of these embodiments, R₄ and R₅ are both F. In the embodiments in which R₄ and R₅ are both F, R₂ may be CH(CH₃)CF₃, CH(isopropyl)CF₃, CH(isopropyl)CH₃, or CH(tert-butyl)CH₃. In other embodiments in which R₄ and R₅ are both F, R₂ and R₃, together with the N atom to which they are attached, may form piperidinyl, azepanyl, or azocanyl.

In certain other embodiments in which R₆ is cyano, R₄ is H and R₅ is F. In some of these embodiments, R₂ may be CH(CH₃)CF₃, CH(isopropyl)CF₃, CH(isopropyl)CH₃, or CH(tert-butyl)CH₃. In some other of these embodiments, R₂ and R₃, together with the N atom to which they are attached, form piperidinyl, azepanyl, or azocanyl.

In other embodiments, R₆ is ethynyl. In some of these embodiments, R₄ and R₅ may both be F. With respect to certain of these embodiments, R₂ may be CH(CH₃)CF₃, CH(isopropyl)CF₃, CH(isopropyl)CH₃, or CH(tert-butyl)CH₃. In certain other of these embodiments, R₂ and R₃, together with the N atom to which they are attached, form piperidinyl, azepanyl, or azocanyl.

In certain other embodiments in which R₆ is ethynyl, R₄ is H and R₅ is F. In some of these embodiments, R₂ is CH(CH₃)CF₃, CH(isopropyl)CF₃, CH(isopropyl)CH₃, or CH(tert-butyl)CH₃. In certain other of these embodiments, R₂ and R₃, together with the N atom to which they are attached, form piperidinyl, azepanyl, or azocanyl.

In some embodiments, R₆ is —C≡C—(CH₂)ₙ—R₇. In certain of these embodiments, n is 1-3. In some of these embodiments, R₄ and R₅ are both F.

Exemplary compounds according to the present disclosure are depicted in Table 1, below:

TABLE 1

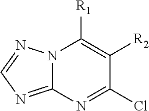

| Compound | | |
|---|---|---|
| Number | R₁ | R₂ |
| 19 | 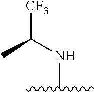 [SMILES: *N[C@@H](C)C(F)(F)F] | 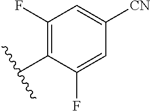 [SMILES: *c1c(F)cc(C#N)cc1F] |
| 61 | 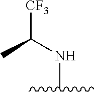 [SMILES: *N[C@@H](C)C(F)(F)F] | 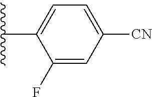 [SMILES: *c1ccc(C#N)cc1F] |
| 62 | 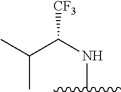 [SMILES: *N[C@H](C(C)C)C(F)(F)F] | 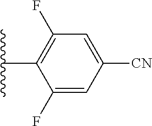 [SMILES: *c1c(F)cc(C#N)cc1F] |
| 63 | 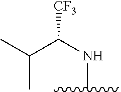 [SMILES: *N[C@H](C(C)C)C(F)(F)F] | 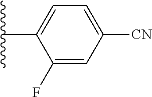 [SMILES: *c1ccc(C#N)cc1F] |
| 66 | 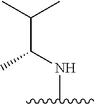 [SMILES: *N[C@H](C)C(C)C] | 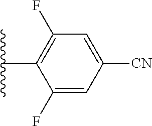 [SMILES: *c1c(F)cc(C#N)cc1F] |
| 67 | 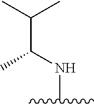 [SMILES: *N[C@H](C)C(C)C] | 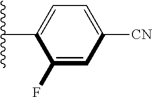 |
| | 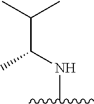 [SMILES: *N[C@H](C)C(C)C] | 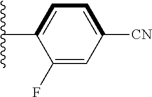 |
| 68 | 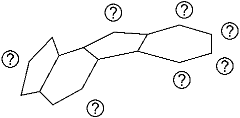 | |
| 69 | 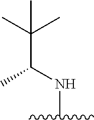 [SMILES: *N[C@H](C)C(C)(C)C] | 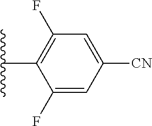 [SMILES: *c1c(F)cc(C#N)cc1F] |
| 70 | 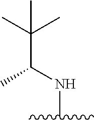 [SMILES: *N[C@H](C)C(C)(C)C] | 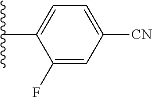 [SMILES: *c1ccc(C#N)cc1F] |
| 73 | 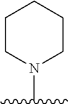 [SMILES: *N1CCCCC1] | 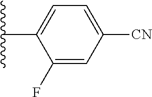 [SMILES: *c1ccc(C#N)cc1F] |
| 74 | 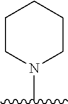 [SMILES: *N1CCCCC1] | 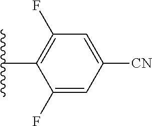 [SMILES: *c1c(F)cc(C#N)cc1F] |
| 75 | 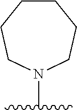 [SMILES: *N1CCCCCC1] | 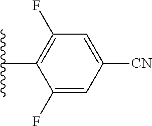 [SMILES: *c1c(F)cc(C#N)cc1F] |
| 76 | 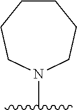 [SMILES: *N1CCCCCC1] | 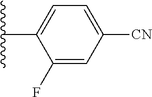 [SMILES: *c1ccc(C#N)cc1F] |
| 77 | 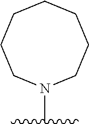 [SMILES: *N1CCCCCCC1] | 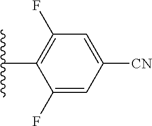 [SMILES: *c1c(F)cc(C#N)cc1F] |
| 78 | 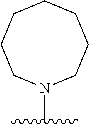 [SMILES: *N1CCCCCCC1] | 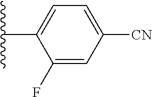 [SMILES: *c1ccc(C#N)cc1F] |

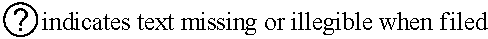

Also provided are compositions for treating a neurodegenerative disease or cancer comprising a therapeutically effective amount of a compound according to any one of the embodiments described above. Also disclosed are methods of treating a neurodegenerative disease or cancer in a subject in need thereof comprising administering to the subject a therapeutically effective amount of a compound according to any one of the embodiments described above.

As used herein, the phrase “therapeutically effective amount” refers to the amount of active compound that elicits the biological or medicinal response that is being sought in a tissue, system, animal, individual or human by a researcher, veterinarian, medical doctor or other clinician, which includes one or more of the following:

(1) at least partially preventing the disease or condition or a symptom thereof; for example, preventing a disease, condition or disorder in an individual who may be predisposed to the disease, condition or disorder but does not yet experience or display the pathology or symptomatology of the disease;

(2) inhibiting the disease or condition; for example, inhibiting a disease, condition or disorder in an individual who is experiencing or displaying the pathology or symptomatology of the disease, condition or disorder (i.e., including arresting further development of the pathology and/or symptomatology); and

(3) at least partially ameliorating the disease or condition; for example, ameliorating a disease, condition or disorder in an individual who is experiencing or displaying the pathology or symptomatology of the disease, condition or disorder (i.e., including reversing the pathology and/or symptomatology).

The compound according to the present disclosure may be provided in a composition that is formulated for any type of administration. For example, the compositions may be formulated for administration orally, topically, parenterally, enterally, or by inhalation. The active compound may be formulated for neat administration, or in combination with conventional pharmaceutical carriers, diluents, or excipients, which may be liquid or solid. The applicable solid carrier, diluent, or excipient may function as, among other things, a binder, disintegrant, filler, lubricant, glidant, compression aid, processing aid, color, sweetener, preservative, suspensing/dispersing agent, tablet-disintegrating agent, encapsulating material, film former or coating, flavoring agent, or printing ink. Any material used in preparing any dosage unit form is preferably pharmaceutically pure and substantially non-toxic in the amounts employed. In addition, the active compound may be incorporated into sustained-release preparations and formulations. Administration in this respect includes administration by, inter alia, the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol, and rectal systemic.

In powders, the carrier, diluent, or excipient may be a finely divided solid that is in admixture with the finely divided active ingredient. In tablets, the active ingredient is mixed with a carrier, diluent or excipient having the necessary compression properties in suitable proportions and compacted in the shape and size desired. For oral therapeutic administration, the active compound may be incorporated with the carrier, diluent, or excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained.

Liquid carriers, diluents, or excipients may be used in preparing solutions, suspensions, emulsions, syrups, elixirs, and the like. The active ingredient of this invention can be dissolved or suspended in a pharmaceutically acceptable liquid such as water, an organic solvent, a mixture of both, or pharmaceutically acceptable oils or fat. The liquid carrier, excipient, or diluent can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Suitable solid carriers, diluents, and excipients may include, for example, calcium phosphate, silicon dioxide, magnesium stearate, talc, sugars, lactose, dextrin, starch, gelatin, cellulose, methyl cellulose, ethylcellulose, sodium carboxymethyl cellulose, microcrystalline cellulose, polyvinylpyrrolidine, low melting waxes, ion exchange resins, croscarmellose carbon, acacia, pregelatinized starch, crospovidone, HPMC, povidone, titanium dioxide, polycrystalline cellulose, aluminum methahydroxide, agar-agar, tragacanth, or mixtures thereof.

Suitable examples of liquid carriers, diluents and excipients, for example, for oral, topical, or parenteral administration, include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil), or mixtures thereof.

For parenteral administration, the carrier, diluent, or excipient can also be an oily ester such as ethyl oleate and isopropyl myristate. Also contemplated are sterile liquid carriers, diluents, or excipients, which are used in sterile liquid form compositions for parenteral administration. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier, diluent, or excipient may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compound in the pharmaceutically appropriate amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and freeze drying techniques that yield a powder of the active ingredient or ingredients, plus any additional desired ingredient from the previously sterile-filtered solution thereof.

Thus, the active compound may be in the present compositions and methods in an effective amount by any of the conventional techniques well-established in the medical field. For example, the administration may be in the amount of about 0.1 mg/day to about 500 mg per day. In some embodiments, the administration may be in the amount of about 250 mg/kg/day. Thus, administration may be in the amount of about 0.1 mg/day, about 0.5 mg/day, about 1.0 mg/day, about 5 mg/day, about 10 mg/day, about 20 mg/day, about 50 mg/day, about 100 mg/day, about 200 mg/day, about 250 mg/day, about 300 mg/day, or about 500 mg/day.

In accordance with the methods of treating a neurodegenerative disease in a subject according to the present disclosure, the neurodegenerative disease may be characterized by a tauopathy or compromised microtubule function in the brain of the subject. In certain embodiments, the neurodegenerative disease may be Alzheimer's disease, frontotemporal lobar degeneration, Pick's disease, progressive supranuclear palsy (PSP), corticobasal degeneration, Parkinson's disease (PD), PD with dementia, Lewy body disease with dementia, or amyotrophic lateral sclerosis. In other embodiments, the neurodegenerative disease is traumatic brain injury or post traumatic stress disorder. The traumatic brain injury may be, for example, repetitive traumatic brain injury or chronic traumatic encephalopathy. In other embodiments, the neurodegenerative disease is schizophrenia.

In accordance with the methods of treating cancer in a subject according to the present disclosure, the cancer may be a carcinoma, sarcoma, melanoma, lymphoma, or leukemia. Common carcinomas include, for example, cancers originating in the skin, lungs, breasts, pancreas, and other organs and glands. Sarcomas are represented by cancers that arise in bone, muscle, fat, blood vessels, cartilage, or other soft or connective tissues of the body. Exemplary cancers that may be treated in accordance with the present methods include brain cancers such as gliomas and astrocytomas, bladder cancer, breast cancer, colon or rectal cancer, endometrial cancer, kidney cancer, leukemia, liverlung cancer, melanoma, non-hodgkin lymphoma, pancreatic cancer, prostate cancer, or thyroid cancer.

Hereinafter, the present disclosure will be described in more detail through Examples, which are intended to be illustrative to the present disclosure, although present disclosure is not limited to the Examples.

### Example 1—Synthetic Procedure

As part of these studies, a total of 73 compounds have been synthesized and tested, including seven previously described triazolopyrimidines; 25 compounds exemplified in the patent literature; and 41 structurally novel congeners. In all cases, the triazolopyrimidine ring was accessed via cyclocondensation reaction between the appropriate diethylmalonate (79-95, Scheme 1) and 1H-1,2,4-triazol-5-amine. Next, treatment with phosphorous oxychloride provided the corresponding 5,7-dichloro triazolopyrimidines (96-112, Scheme 1). Chemoselective amination at C7 with (S)-1,1,1-trifluoropropan-2-amine furnished triazolopyrimidines 6-22 (Scheme 1).

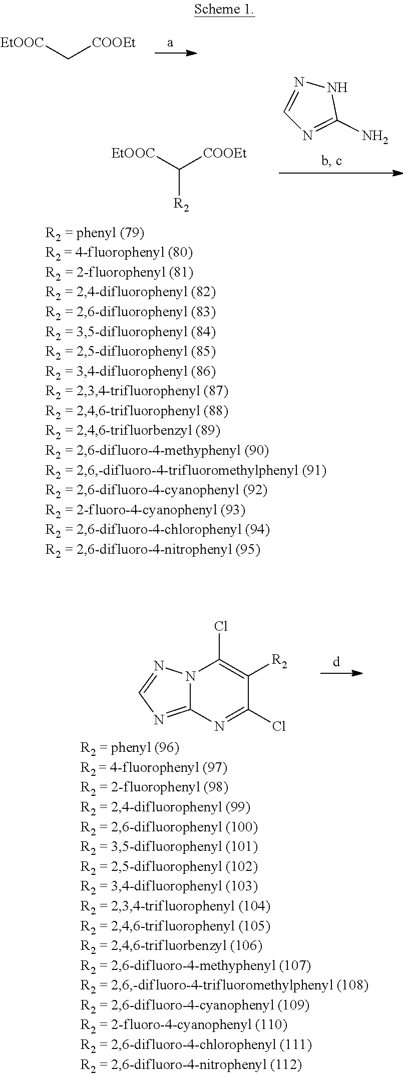

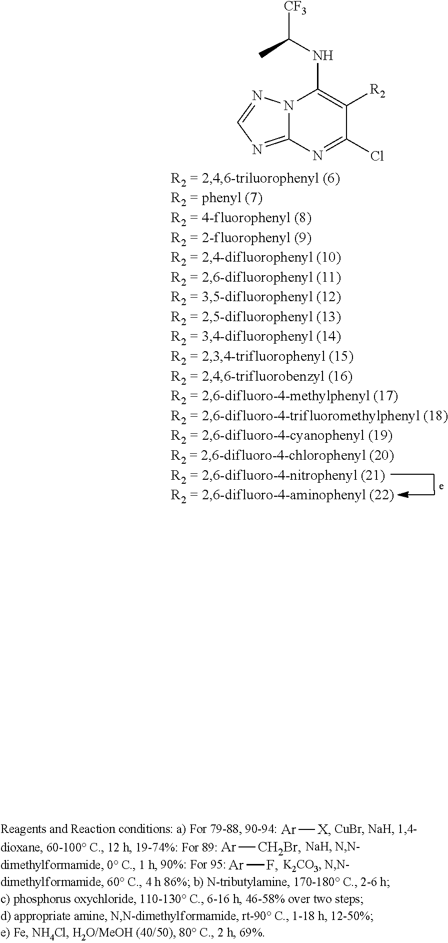

The synthesis of compounds 61-78 (Scheme 5), that relative to the control compound 6 are modified at both fragments at C6 and C7, was conducted by reacting the appropriate triazolopyrimidine dichlorides (i.e., 98, 99, 109, or 110) with amine fragments (Scheme 5). In selected cases (i.e., 67 and 68) purification via silica gel column chromatography afforded individual atropoisomers, whose structures could be determined by X-ray crystallography (Scheme 5):

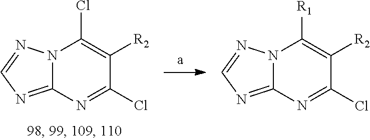

| Cpd # | R₁ | R₂ |
|---|---|---|
| 61 | 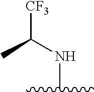 [SMILES: *N[C@@H](C)C(F)(F)F] | 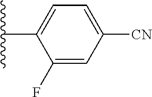 [SMILES: *c1ccc(C#N)cc1F] |
| 62 | 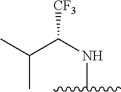 [SMILES: *N[C@H](C(C)C)C(F)(F)F] | 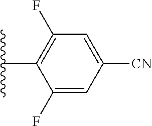 [SMILES: *c1c(F)cc(C#N)cc1F] |
| 63 | 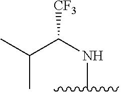 [SMILES: *N[C@H](C(C)C)C(F)(F)F] | 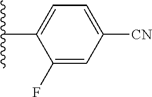 [SMILES: *c1ccc(C#N)cc1F] |
| 64 | 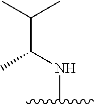 [SMILES: *N[C@H](C)C(C)C] | 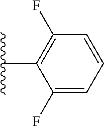 [SMILES: *c1c(F)cccc1F] |
| 65 | 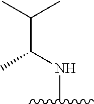 [SMILES: *N[C@H](C)C(C)C] | 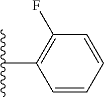 [SMILES: *c1ccccc1F] |
| 66 | 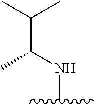 [SMILES: *N[C@H](C)C(C)C] | 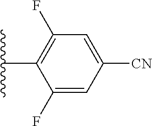 [SMILES: *c1c(F)cc(C#N)cc1F] |
| 67 | 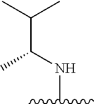 [SMILES: *N[C@H](C)C(C)C] | 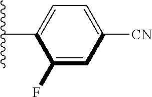 |
| | 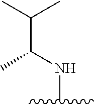 [SMILES: *N[C@H](C)C(C)C] | 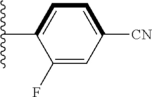 |
| 68 | 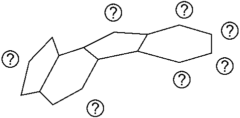 | |
| 69 | 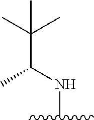 [SMILES: *N[C@H](C)C(C)(C)C] | 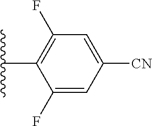 [SMILES: *c1c(F)cc(C#N)cc1F] |
| 70 | 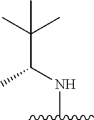 [SMILES: *N[C@H](C)C(C)(C)C] | 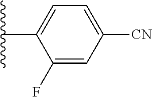 [SMILES: *c1ccc(C#N)cc1F] |
| 71 | 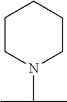 | 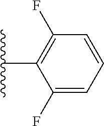 [SMILES: *c1c(F)cccc1F] |
| 72 | 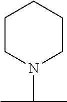 | 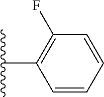 [SMILES: *c1ccccc1F] |
| 73 | 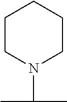 | 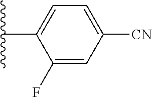 [SMILES: *c1ccc(C#N)cc1F] |
| 74 | 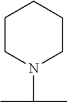 | 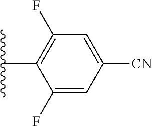 [SMILES: *c1c(F)cc(C#N)cc1F] |
| 75 | 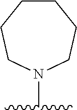 [SMILES: *N1CCCCCC1] | 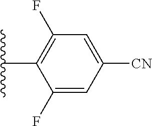 [SMILES: *c1c(F)cc(C#N)cc1F] |
| 76 | 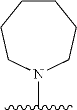 [SMILES: *N1CCCCCC1] | 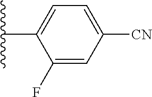 [SMILES: *c1ccc(C#N)cc1F] |
| 77 | 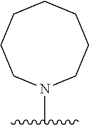 [SMILES: *N1CCCCCCC1] | 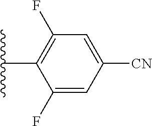 [SMILES: *c1c(F)cc(C#N)cc1F] |
| 78 | 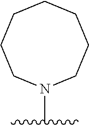 [SMILES: *N1CCCCCCC1] | 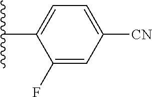 [SMILES: *c1ccc(C#N)cc1F] |

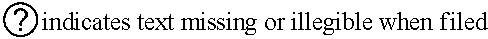

Scheme 5. Reagents and Reaction conditions: a) appropriate amine, N,N-dimethylformamide, rt, 1 h, 56-86%.

### Example 2—Materials and Methods

All solvents were reagent grade. All reagents were purchased from Aldrich or Acros and used as received. Thin layer chromatography (TLC) was performed with 0.25 mm E. Merck precoated silica gel plates. Silica gel column chromatography was performed with silica gel 60 (particle size 0.040-0.062 mm) supplied by Silicycle and Sorbent Technologies. TLC spots were detected by viewing under a UV light. Melting points (mp) were acquired on a Mel-Temp II (model. 1001) and are uncorrected. Infrared (IR) spectra were recorded on a Bruker, model Alpha spectrometer (part number 1003271/03). Proton (H) and carbon (¹³C) NMR spectra were recorded on a 500 MHz Bruker AMX-500 spectrometer or 600 MHz Bruker Avance III spectrometer. Chemical shifts were reported relative to solvents. High-resolution mass spectra were measured using an Agilent 6230 time-of-flight mass spectrometer (TOFMS) with Jet stream electrospray ionisation source (ESI). Single-crystal X-ray structure determinations were performed Bruker MicroStar with an APEX II detector, double-bounce micro-focus optics and a Cu rotating anode source. Analytical reverse-phase (Sunfire C18; 4.6 mm×50 mm, 5 mL) high-performance liquid chromatography (HPLC) was performed with a Gilson HPLC equipped with UV and mass detector. All samples were analyzed employing a linear gradient from 10% to 90% of CH₃CN in water over 8 min and flow rate of 1 mL/min, and unless otherwise stated, the purity level was >95%. Preparative reverse-phase HPLC purifications were performed on a Gilson instrument employing Waters SunFire preparative Cis OBD columns (5 μm 19 mm×50 mm or 19 mm×100 mm). Purifications were carried out employing a linear gradient from 10% to 90% of CH₃CN in water for 15 min with a flow rate of 20 mL/min. Unless otherwise stated, all final compounds were found to be >95% pure as determined by HPLC/MS and NMR.

### General Procedure A (Synthesis of Diethylmalonate Derivatives):

To a suspension of NaH (60 wt % in mineral oil) (1.00 equiv) in anhydrous 1,4-dioxane (previously degassed with N₂) (1.75 mol/L from aryl bromide derivate) at 60° C. under N₂ was slowly added diethyl malonate (3.00 equiv) and stirred for 10 minutes. Then, CuBr (1.20 equiv) and aryl bromide derivate were added and the reaction was heated at reflux overnight. Then, at r.t the reaction was quenched with HCl 12N (1.40 equiv). The mixture was filtrated and washed with H₂O. The filtrate was extracted with EtOAc (×3). The combined organic extracts were washed with brine, then dried over MgSO₄, filtered and concentrated in vacuo. The crude products were purification by silica gel column chromatography or by preparative reverse-phase HPLC to obtain the pure diethylmalonate derivatives.

### General Procedure B (Synthesis of Dichloro-Triazolopyrimidine Derivatives):

A mixture of diethylmalonate derivate (1.00 equiv), 3-amino-1,2,4-triazole (1.05 equiv) and tributylamine (1.05 equiv) in sealed tube was stirred at 170° C. for 2 h. After cooling at 130° C., toluene was added (1.00 mol/L from diethylmalonate derivative). Then, the reaction was cooled at 50° C. and an aqueous solution of NaOH (50% wt) (160 μL/mmol from diethylmalonate derivate) was added. After that, the mixture was stirred at 0° C. for 10 minute and filtered. The solid formed was washed with cooled toluene and dried. This disodic derivate (1.00 equiv) and POCl₃ (17.80 equiv) were mixed in a sealed tube and heated at 130° C. for 6 h. Then, the reaction was quenched with H₂O and extracted with EtOAc (×2). The combined organic extracts were washed with water (×5), brine, dried over MgSO₄, filtered and concentrated in vacuo. The resulting product was directly used in the next reaction without further purification.

### General Procedure C (Addition of the Amine):

According to a reported procedure (Zhang et al., J. Med. Chem., 2007, 50, 319-327), to 5,7-dichloro-6-(2,4,6-trifluorophenyl)-[1,2,4]triazolo[1,5-a]pyrimidine (1.0 equiv) in DMF (0.1 M) at room temperature was added i-Pr₂NEt (3.0 equiv) or Et₃N (3.0 equiv) if necessary and the appropriate amine (1.5 to 3.0 equiv). The reaction mixture was stirred for 0.5-16 h and diluted with H₂O. The organic layer was washed with a 1N solution of hydrochloric acid (2×), the aqueous phase was extracted with EtOAc (3×), and the combined organic layers were washed with brine (2×), dried (MgSO₄), filtered, and concentrated. The products were purified by flash chromatography or reverse-phase HPLC.

### General Procedure D (Addition of the Side Chain):

According to reported procedures (Zhang et al., J. Med. Chem., 2007, 50, 319-327; Zhang et al., Bioorg. Med. Chem. 2009, 17, 111-8) to a suspension of NaH (4.0 equiv) in a 2:1 mixture of DMSO and THF (0.35 M) was added the aminoalcohol (4.0 equiv), and the mixture was heated to 60° C. for 1 h. The resulting solution was treated with a solution of trifluoroarene (1.0 equiv) in a 1:1 mixture DMSO and THF (0.5 M). The reaction mixture was stirred at 60° C. for 3 h and monitored by LCMS. If the starting material remained after 3 h, additional aminoalcohol (4.0 equiv) and NaH (4.0 equiv) were added, sequentially, and the reaction mixture was heated for 16 h. Following complete consumption of the starting material, the reaction mixture was cooled to room temperature and diluted with H₂O and EtOAc. The organic layer was washed with H₂O and brine, and the combined aqueous layers were extracted with EtOAc (×3). The combined organic layers were dried (MgSO₄), filtered, and concentrated. The crude products were purified by reverse-phase HPLC.

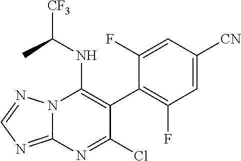 [SMILES: C[C@H](Nc1c(-c2c(F)cc(C#N)cc2F)c(Cl)nc2ncnn12)C(F)(F)F]

(19)

### (S)-4-(5-Chloro-7-((1,1,1-trifluoropropan-2-yl)amino)-[1,2,4]triazolo[1,5-a]pyrimidin-6-yl)-3,5-difluorobenzonitrile

Following general procedure C using 4-(5,7-dichloro-[1,2,4]triazolo[1,5-a]pyrimidin-6-yl)-3,5-difluorobenzonitrile (30 mg, 0.094 mmol) and (S)-1,1,1-trifluoropropan-2-amine (22 mg, 0.190 mmol). Purification by silica gel column chromatography (0-40% EtOAc in Hexanes) provided the title compound as a white powder (20 mg, 0.050 mmol, 54%).

¹H NMR (600 MHz, CDCl₃) δ 8.41 (s, 1H), 7.44 (t, J=7.1 Hz, 2H), 6.00 (d, J=10.7 Hz, 1H), 4.77 (s, 1H), 1.45 (d, J=6.9 Hz, 3H) ppm.

¹³C NMR (150 MHz, CDCl₃) δ 161.28 (dd, J=256.4, 5.9 Hz), 160.87 (dd, J=254.7, 6.0 Hz), 156.84, 155.68, 154.32, 124.46 (q, J=282.1 Hz), 116.58 (t, J=11.8 Hz), 116.47 (ddd, J=67.6, 25.4, 4.1 Hz), 115.79 (d, J=3.5 Hz), 114.93 (t, J=20.0 Hz), 90.61, 51.27 (q, J=32.2 Hz), 29.83, 15.14 ppm.

IR: 2923, 2239, 1617, 1556, 1141 cm⁻¹

HRMS (ES+) calculated for C₁₅H₉N₆ClF₅ [M+H]⁺ 403.0492, found 403.0486.

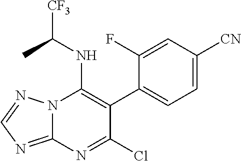 [SMILES: C[C@H](Nc1c(-c2ccc(C#N)cc2F)c(Cl)nc2ncnn12)C(F)(F)F]

(61)

### (S)-4-(5-Chloro-7-((1,1,1-trifluoropropan-2-yl)amino)-[1,2,4]triazolo[1,5-a]pyrimidin-6-yl)-3-fluorobenzonitrile

Following general procedure C using 4-(5,7-dichloro-[1,2,4]triazolo[1,5-a]pyrimidin-6-yl)-3-fluorobenzonitrile (40 mg, 0.13 mmol) and (S)-1,1,1-trifluoropropan-2-amine (31 mg, 0.27 mmol). Purification by reversed phase HPLC provided the title compound as a white powder (31 mg, 0.070 mmol, 54%).

Mixture of Atropoisomers

¹H NMR (600 MHz, CDCl₃) δ 8.41 (s, 1H), 7.68 (ddd, J=7.8, 3.8, 1.5 Hz, 1H), 7.61 (ddd, J=8.4, 3.8, 1.5 Hz, 1H), 7.57 (t, J=7.4 Hz, 1H), 7.50 (t, J=7.3 Hz, 1H), 5.94 (d, J=11.0 Hz, 1H), 5.57 (d, J=10.8 Hz, 1H), 4.97 (s, 1H), 4.50 (s, 1H), 1.40 (d, J=6.9 Hz, 3H) ppm.

¹³C NMR (150 MHz, CDCl₃) δ 161.29 (d, J=52.0 Hz), 159.61 (d, J=50.4 Hz), 156.65 (d, J=24.9 Hz), 155.72 (d, J=11.0 Hz), 154.20 (d, J=57.5 Hz), 145.65 (d, J=62.8 Hz), 135.00 (d, J=2.0 Hz), 133.84 (d, J=2.2 Hz), 129.21 (d, J=4.2 Hz), 124.97 (dd, J=16.2, 13.1 Hz), 124.67 (qd, J=282.3, 34.7 Hz), 120.62 (dd, J=74.3, 25.2 Hz), 116.67 (d, J=2.8 Hz), 116.33 (dd, J=9.3, 5.2 Hz), 97.66, 96.61, 51.26 (dq, J=37.9, 31.8 Hz), 15.13, 14.99 ppm.

HRMS (ES+) calculated for C₁₅H₁₀ClF₄N₆ [M+H]⁺ 385.0586, found 385.0589.

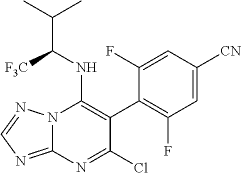 [SMILES: CC(C)[C@@H](Nc1c(-c2c(F)cc(C#N)cc2F)c(Cl)nc2ncnn12)C(F)(F)F]

(62)

### (R)-4-(5-chloro-7-((1,1,1-trifluoro-3-methylbutan-2-yl)amino)-[1,2,4]triazolo[1,5-a]pyrimidin-6-yl)-3,5-difluorobenzonitrile

Following general procedure C using 4-(5,7-dichloro-[1,2,4]triazolo[1,5-a]pyrimidin-6-yl)-3,5-difluorobenzonitrile (55 mg, 0.17 mmol) and (R)-1,1,1-trifluoro-3-methyl-2-butylamine (48 mg, 0.34 mmol). Purification by reversed phase HPLC provided the title compound as a white powder (26 mg, 0.060 mmol, 36%).

¹H NMR (600 MHz, CDCl₃, mixture of atropoisomers) δ 8.81 (s, 0.5H), 8.69 (s, 0.5H), 7.46 (t, J=9 Hz, 2H), 7.33 (d, J=12 Hz, 1H), 6.28 (bs, 1H), 4.00 (bs, 1H), 2.19 (hept, J=6 Hz, 1H), 1.04 (d, J=6 Hz, 1H), 0.97 (d, J=6 Hz, 1H) ppm.

¹³C NMR (150 MHz, CDCl₃ mixture of atropoisomers) δ 162.05, 162.01, 161.79, 161.74, 161.54, 161.49, 160.35, 160.30, 160.10, 160.07, 159.86, 159.814, 158.37, 157.23, 157.16, 155.65, 154.61, 152.15, 146.27, 144.77, 139.79, 133.47, 124.33 (q, J=283.88 Hz), 117.41, 116.85, 116.82, 116.78, 116.70, 116.68, 116.65, 116.40, 116.17, 116.00, 115.84, 115.30, 115.04, 114.91, 113.00, 109.71, 106.86, 59.28 (q, J=28.69 Hz), 28.28, 19.80, 16.60 ppm.

HRMS (ES+) calculated for C₁₈H₁₇ClFN₆ [M+H]⁺ 429.0659, found 429.0655.

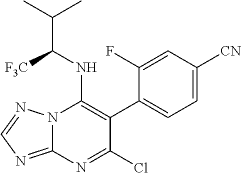 [SMILES: CC(C)[C@@H](Nc1c(-c2ccc(C#N)cc2F)c(Cl)nc2ncnn12)C(F)(F)F]

(63)

### (R)-4-(5-chloro-7-((1,1,1-trifluoro-3-methylbutan-2-yl)amino)-[1,2,4]triazolo[1,5-a]pyrimidin-6-yl)-3-fluorobenzonitrile

Following general procedure C using 4-(5,7-dichloro-[1,2,4]triazolo[1,5-a]pyrimidin-6-yl)-3-fluorobenzonitrile (51 mg, 0.17 mmol) and (R)-1,1,1-trifluoro-3-methyl-2-butylamine (47 mg, 0.34 mmol). Purification by reversed phase HPLC provided the title compound as a white powder (13 mg, 0.060 mmol, 19%).

¹H NMR (600 MHz, CDCl₃, mixture of atropoisomers) δ 8.42 (s, 0.5H), 7.69 (d, J=7.9 Hz, 0.5H), 7.63 (d, J=8.2 Hz, 0.5H), 7.60 (d, J=8.2 Hz, 0.5H), 7.55 (t, J=7.5 Hz, 0.5H), 7.48 (t, J=7.3 Hz, 0.5H), 4.86 (d, J=10.2 Hz, 0.5H), 4.40 (d, J=4.1 Hz, 0.5H), 2.14 (hept, J=6.9 Hz, 1H), 1.02 (d, J=6.1 Hz, 3H), 0.97 (d, J=6.7 Hz, 3H).

¹³C NMR (150 MHz, CDCl₃, mixture of atropoisomers) δ 160.44 (dd, J=253.68, 28.69 Hz), 158.91, 155.66, 155.60, 134.88, 134.50, 129.18, 129.02, 126.68, 124.86, 120.97, 120.81, 120.39, 120.21, 116.74 (t, J=3.0 Hz), 116.42 (t, J=13.4 Hz) 116.36 (t, J=13.4 Hz) 59.08 (q, J=28.7 Hz), 55.89 (q, J=28.7 Hz), 28.33, 28.19, 27.75, 19.92, 19.80, 17.11, 16.74, 16.69 ppm.

HRMS (ES+) calculated for C₁₈H₁₇ClFN₆ [M+H]⁺ 411.0754, found 411.0747.

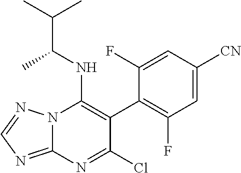 [SMILES: CC(C)[C@@H](C)Nc1c(-c2c(F)cc(C#N)cc2F)c(Cl)nc2ncnn12]

(66)

### (R)-4-(5-chloro-7-((3-methylbutan-2-yl)amino)-[1,2,4]triazolo[1,5-a]pyrimidin-6-yl)-3,5-difluorobenzonitrile

Following general procedure C using 4-(5,7-dichloro-[1,2,4]triazolo[1,5-a]pyrimidin-6-yl)-3,5-difluorobenzonitrile (30 mg, 0.092 mmol) and (R)-3-methylbutan-2-amine (17 mg, 0.190 mmol). Purification by reversed phase HPLC provided the title compound as a white powder (28 mg, 0.074 mmol, 81%).

¹H NMR (600 MHz, DMSO-d₆) δ 8.62 (s, 1H), 8.11 (t, J=7.8 Hz, 2H), 7.88 (s, 1H), 1.78 (dq, J=13.9, 6.8 Hz, 1H), 1.10 (d, J=6.6 Hz, 3H), 0.74 (dd, J=6.8, 2.9 Hz, 6H).

¹³C NMR (151 MHz, DMSO-d₆) δ 160.71 (dd, J=249.2, 6.8 Hz), 160.38 (dd, J=249.5, 6.3 Hz), 155.02, 146.81, 117.00 (ddd, J=26.3, 12.6, 3.9 Hz), 116.48 (d, J=3.5 Hz), 114.83 (t, J=12.8 Hz).

IR: 2966, 2220, 1690, 1562, 1422, 1204 cm⁻¹

HRMS (ES+) calculated for C₁₇H₁₆ClF₂N₆ [M+H]⁺ 377.1088, found 377.1089.

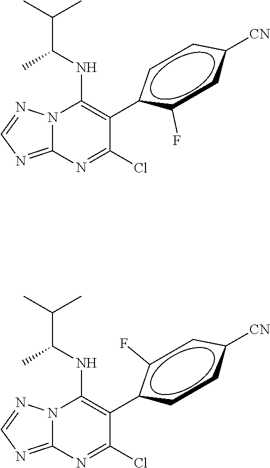

(67), (68)

### (R)-4-(5-chloro-7-((3-methylbutan-2-yl)amino)-[1,2,4]triazolo[1,5-a]pyrimidin-6-yl)-3-fluorobenzonitrile

Following general procedure C using 4-(5,7-dichloro-[1,2,4]triazolo[1,5-a]pyrimidin-6-yl)-3-fluorobenzonitrile (48 mg, 0.15 mmol) and (R)-3-methylbutan-2-amine (37 mg, 0.42 mmol). The mixture was dried in vacuo and purified by silica gel column chromatography to furnish atropoisomer A as a white solid (29 mg, 0.081 mmol, 39%) and atroposiomer B (23 mg, 0.064 mmol, 30%).

### Atroposisomer A

¹H NMR (600 MHz, CDCl₃) δ 8.35 (s, 1H), 7.63 (dd, J 12.0, 3.0 Hz, 1H), 7.56 (dd, J 12.0, 3.0 Hz, 1H), 7.53 (t, J=9.0 Hz, 1H), 6.29 (d, J=12.0 Hz, 1H), 3.00 (bs, 1H), 1.64 (oct, J=6.0 Hz, 1H), 0.99 (d, J=6 Hz, 3H), 0.80 (d, J=6 Hz, 3H), 0.77 (d, J=6 Hz, 3H) ppm.

¹³C NMR (150 MHz, CDCl₃) δ 161.37, 159.70, 156.91, 155.15, 153.15, 145.67, 134.39, 128.71, 128.69, 127.13 (d, J=16.6 Hz), 120.02 (d, J=25.7 Hz), 116.98, 115.50 (d, J=9.1 Hz), 94.53, 55.04, 33.48, 18.16, 17.80, 17.14 ppm.

### Atropoisomer B

¹H NMR (600 MHz, CDCl₃) δ 8.35 (s, 1H), 7.63 (dd, J 12.0, 3.0 Hz, 1H), 7.56 (dd, J 12.0, 3.0 Hz, 1H), 7.52 (t, J=9.0 Hz, 1H), 6.30 (bs, 1H), 3.05 (bs, 1H), 1.61 (oct, J=6.0 Hz, 1H), 1.05 (d, J=6 Hz, 3H), 0.79 (d, J=6 Hz, 3H), 0.75 (d, J=6 Hz, 3H) ppm.

¹³C NMR (150 MHz, CDCl₃) δ 161.40, 159.73, 156.90, 155.19, 153.57, 145.67, 135.08, 134.40 (d, J 3.0 Hz), 128.55 (d, J 4.5 Hz), 127.03 (d, J=15.1 Hz), 120.22 (d, J=25.7 Hz), 117.01, 115.45 (d, J=9.1 Hz), 94.37, 54.85, 33.80, 18.20, 17.90, 17.82 ppm.

IR: 2963, 2220, 1608, 1570, 1260, 1156 cm⁻¹

HRMS (ES+) calculated for C₁₈H₁₇ClFN₆ [M+H]⁺ 359.1182, found 359.1184.

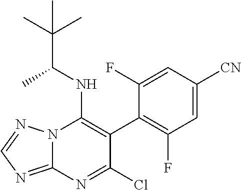 [SMILES: C[C@@H](Nc1c(-c2c(F)cc(C#N)cc2F)c(Cl)nc2ncnn12)C(C)(C)C]

(69)

### (R)-4-(5-Chloro-7-((3,3-dimethylbutan-2-yl)amino)-[1,2,4]triazolo[1,5-a]pyrimidin-6-yl)-3,5-difluorobenzonitrile

Following general procedure C using 4-(5,7-dichloro-[1,2,4]triazolo[1,5-a]pyrimidin-6-yl)-3,5-difluorobenzonitrile (20 mg, 0.060 mmol) and (R)-3,3-dimethylbutan-2-amine (13 mg, 0.13 mmol). Purification by reversed phase HPLC provided the title compound as a white powder (18 mg, 0.046 mmol, 75%).

¹H NMR (600 MHz, CDCl₃) δ 8.36 (s, 1H), 7.45-7.41 (m, 2H), 6.52 (d, J=10.8 Hz, 1H), 2.94 (s, 1H), 1.02 (d, J=6.7 Hz, 3H), 0.84 (s, 9H) ppm.

¹³C NMR (150 MHz, CDCl₃) δ 161.33 (dd, J=253.8, 6.2 Hz), 161.05 (dd, J=253.8, 6.2 Hz), 157.12, 155.22, 146.03, 116.22 (d, J=4.0 Hz), 116.13-115.95 (m), 115.95-115.82 (m), 58.57, 34.93, 25.86, 16.56 ppm.

HRMS (ES+) calculated for C₁₈H₁₈ClF₂N₆ [M+H]⁺ 391.1244, found 391.1241.

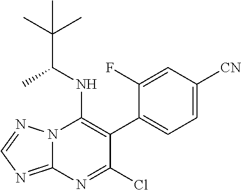 [SMILES: C[C@@H](Nc1c(-c2ccc(C#N)cc2F)c(Cl)nc2ncnn12)C(C)(C)C]

(70)

### (R)-4-(5-Chloro-7-((3,3-dimethylbutan-2-yl)amino)-[1,2,4]triazolo[1,5-a]pyrimidin-6-yl)-3-fluorobenzonitrile

Following general procedure C using 4-(5,7-dichloro-[1,2,4]triazolo[1,5-a]pyrimidin-6-yl)-3-fluorobenzonitrile (40 mg, 0.13 mmol) and (R)-3,3-dimethylbutan-2-amine (28 mg, 0.27 mmol). Purification by reversed phase HPLC provided the title compound as a white powder (27 mg, 0.072 mmol, 56%).

### Mixture of Atropoisomers

¹H NMR (600 MHz, CDCl₃) δ 8.41-8.34 (m, 1H), 7.67-7.61 (m, 1H), 7.60-7.55 (m, 1H), 7.55-7.49 (m, 1H), 6.37 (s, 1H), 2.94 (m, 1H), 1.01 (d, J=6.7 Hz, 1H), 0.94 (d, J=6.7 Hz, 2H), 0.82 (s, 5H), 0.81 (s, 4H) ppm.

¹³C NMR (150 MHz, CDCl₃) δ 161.71-159.28 (m), 156.76, 156.67, 145.96, 135.28 (d, J=2.4 Hz), 133.96 (d, J=2.3 Hz), 128.65 (d, J=4.1 Hz), 128.43 (d, J=4.1 Hz), 119.96 (dd, J=37.6, 25.4 Hz), 116.86 (d, J=2.8 Hz), 115.42 (dd, J=16.7, 9.3 Hz), 94.27, 94.14, 58.27, 58.07, 35.01, 34.82, 25.77 (d, J=1.9 Hz), 16.42 (d, J=2.2 Hz) ppm.

HRMS (ES+) calculated for C₁₈H₁₉ClFN₆ [M+H]⁺ 373.1338, found 373.1338.

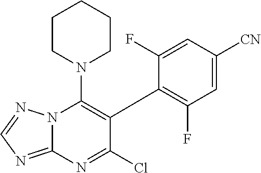 [SMILES: N#Cc1cc(F)c(-c2c(Cl)nc3ncnn3c2N2CCCCC2)c(F)c1]

(74)

### 4-(5-chloro-7-(piperidin-1-yl)-[1,2,4]triazolo[1,5-a]pyrimidin-6-yl)-3,5-difluorobenzonitrile

Following general procedure C using 4-(5,7-dichloro-[1,2,4]triazolo[1,5-a]pyrimidin-6-yl)-3,5-difluorobenzonitrile (48 mg, 0.15 mmol) and piperidine (25 mg, 0.30 mmol). Purification by reversed phase HPLC provided the title compound as a white powder (5 mg, 0.010 mmol, 9%).

¹³C NMR (150 MHz, CDCl₃) δ 160.71 (dd, J=253.7, 9 Hz), 157.05, 155.60, 155.44, 150.84, 118.10 (t, J=15.1 Hz), 116.22 (d, J=6 Hz), 116.04, 115.06 (t, J=12.1 Hz), 96.75, 51.34, 25.92, 23.57 ppm.

HRMS (ES+) calculated for C₁₈H₁₇ClFN₆ [M+H]⁺ 375.0931, found 375.0931.

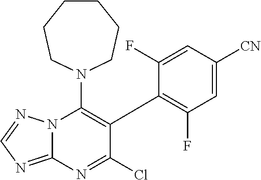 [SMILES: N#Cc1cc(F)c(-c2c(Cl)nc3ncnn3c2N2CCCCCC2)c(F)c1]

(75)

### 4-(7-(Azepan-1-yl)-5-chloro-[1,2,4]triazolo[1,5-a]pyrimidin-6-yl)-3,5-difluorobenzonitrile (51)

Following general procedure C using 4-(5,7-dichloro-[1,2,4]triazolo[1,5-a]pyrimidin-6-yl)-3,5-difluorobenzonitrile (20 mg, 0.060 mmol) and azepane (13 mg, 0.13 mmol). Purification by reversed phase HPLC provided the title compound as a white powder (15 mg, 0.039 mmol, 63%).

¹H NMR (600 MHz, CDCl₃) δ 8.38 (s, 1H), 7.40 (d, J=6.0 Hz, 2H), 3.45-3.39 (m, 4H), 1.78-1.70 (m, 4H), 1.64-1.60 (m, 4H) ppm.

¹³C NMR (150 MHz, CDCl₃) δ 160.74 (dd, J=253.4, 6.6 Hz), 157.14, 155.26, 152.10, 118.61 (t, J=19.7 Hz), 116.30-115.97 (m), 115.12 (t, J=11.9 Hz), 97.32, 53.99, 51.22, 28.27, 28.17, 27.28, 27.07 ppm.

IR: 2928, 2857, 2238, 1589, 1515, 1422 cm⁻¹

HRMS (ES+) calculated for C₁₈H₁₆ClF₂N₆ [M+H]⁺ 389.1088, found 389.1085.

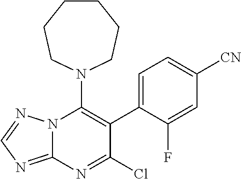 [SMILES: N#Cc1ccc(-c2c(Cl)nc3ncnn3c2N2CCCCCC2)c(F)c1]

(76)

### 4-(7-(Azepan-1-yl)-5-chloro-[1,2,4]triazolo[1,5-a]pyrimidin-6-yl)-3-fluorobenzonitrile (55)

Following general procedure C using 4-(5,7-dichloro-[1,2,4]triazolo[1,5-a]pyrimidin-6-yl)-3-fluorobenzonitrile (40 mg, 0.13 mmol) and azepane (27 mg, 0.27 mmol). Purification by reversed phase HPLC provided the title compound as a white powder (27 mg, 0.073 mmol, 56%).

¹H NMR (600 MHz, CDCl₃) δ 8.34 (s, 1H), 7.61 (dd, J=7.9, 1.6 Hz, 1H), 7.53 (dd, J=8.7, 1.6 Hz, 1H), 7.48 (t, J=7.5 Hz, 1H), 3.38-3.33 (m, 4H), 1.71 (dq, J=8.0, 4.2 Hz, 4H), 1.60 (p, J=3.0 Hz, 4H) ppm.

¹³C NMR (150 MHz, CDCl₃) δ 160.85, 159.18, 156.93, 155.27, 155.18, 151.61, 134.44 (d, J=2.8 Hz), 128.60 (d, J=3.9 Hz), 128.48, 120.11, 119.94, 117.09 (d, J=2.8 Hz), 114.68 (d, J=9.2 Hz), 103.59, 54.05, 28.27, 28.03 ppm.

IR: 2926, 2235, 1589, 1519, 1446 cm⁻¹

HRMS (ES+) calculated for C₁₈H₁₇ClFN₆ [M+H]⁺ 371.1182, found 371.1180.

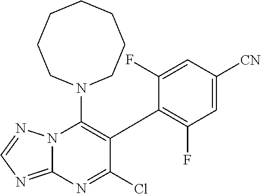 [SMILES: N#Cc1cc(F)c(-c2c(Cl)nc3ncnn3c2N2CCCCCCC2)c(F)c1]

(77)

### 4-(7-(azocan-1-yl)-5-chloro-[1,2,4]triazolo[1,5-a]pyrimidin-6-yl)-3,5-difluorobenzonitrile

Following general procedure C using 4-(5,7-dichloro-[1,2,4]triazolo[1,5-a]pyrimidin-6-yl)-3,5-difluorobenzonitrile (54 mg, 0.17 mmol) and azocane (19 mg, 0.17 mmol). The mixture was dried in vacuo and purified by silica gel column chromatography to furnish the title compound as a white solid (31 mg, 0.077 mmol, 46%).

¹H NMR (600 MHz, CDCl₃) δ 8.29 (s, 1H), 7.30 (d, J=6.0 Hz, 2H), 3.36 (t, J=5.7 Hz, 5H), 1.62 (t, J=5.8 Hz, 5H), 1.52 (t, J=5.5 Hz, 6H), 1.44 (d, J=6.0 Hz, 2H).

¹³C NMR (151 MHz, CDCl₃) δ 160.68 (dd, J=253.6, 6.5 Hz), 157.23, 155.73, 155.12, 151.06, 118.55 (t, J=19.8 Hz), 116.38, 116.34, 116.23, 116.19, 115.23 (t, J=11.8 Hz), 97.29, 52.24, 27.72, 26.80, 24.44 ppm.

HRMS (ES+) calculated for C₁₈H₁₇ClFN₆ [M+H]⁺ 403.1244, found 403.1240.

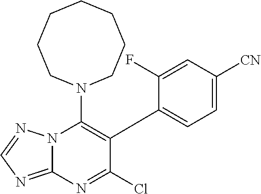 [SMILES: N#Cc1ccc(-c2c(Cl)nc3ncnn3c2N2CCCCCCC2)c(F)c1]

(78)

### 4-(7-(azocan-1-yl)-5-chloro-[1,2,4]triazolo[1,5-a]pyrimidin-6-yl)-3-fluorobenzonitrile

Following general procedure C using 4-(5,7-dichloro-[1,2,4]triazolo[1,5-a]pyrimidin-6-yl)-3-fluorobenzonitrile (48 mg, 0.15 mmol) and azocane (35 mg, 0.30 mmol). The mixture was dried in vacuo and purified by silica gel column chromatography to furnish the title compound as a white solid (32 mg, 0.083 mmol, 53%).

¹H NMR (600 MHz, CDCl₃, mixture of atropoisomers) δ 8.59 (s, 0.4H), 8.42 (s, 0.3H), 7.80-7.77 (m, 0.6H), 7.30 (d, J=6 Hz, 0.3H), 7.16 (d, J=12 Hz, 0.4H), 3.41 (t, J=6 Hz, 1.2H), 3.32 (bs, 1.8H), 3.04 (bs, 1.8H), 1.70-1.41 (m, 10H) ppm.

¹³C NMR (150 MHz, CDCl₃) δ 163.68, 159.94 (dd, J=250.7, 19.6 Hz), 157.21, 155.29, 154.79, 151.33, 150.39, 148.23, 145.52, 144.37, 142.0, 140.38, 134.19, 133.98, 133.96, 132.10, 130.25, 130.20, 129.25, 128.98, 128.89, 128.86, 128.51, 128.40, 128.33, 128.30, 128.26, 128.21, 126.15, 120.38, 120.30, 120.22, 120.13, 117.09, 116.88, 114.89, 114.83, 114.73, 114.67, 103.26, 99.78, 54.20, 52.27, 51.56, 50.31, 44.80, 27.71, 26.84, 25.57, 25.70, 24.27, 23.95, 23.42, 22.69, 22.55, 22.51, 21.84, 21.52 ppm.

HRMS (ES+) calculated for C₁₈H₁₇ClFN₆ [M+H]⁺ 385.1338, found 385.1332.

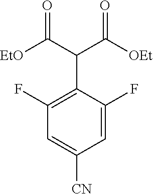 [SMILES: CCOC(=O)C(C(=O)OCC)c1c(F)cc(C#N)cc1F]

(92)

### Diethyl 2-(4-cyano-2,6-difluorophenyl)malonate

A mixture of 3,4,5-trifluorobenzonitrile (1.00 g, 6.37 mmol, 1 equiv), potassium carbonate (1.76 g, 12.7 mmol, 2.00 equiv), and diethyl malonate (1.03 g, 6.43 mmol, 1.01 equiv) in anh. DMF (6.37 ml) under N₂ was stirred at 65° C. until starting material was consumed as indicated by TLC. The reaction mixture was cooled to r.t., washed with 1M HCl (50 mL), and extracted with EtOAc (3×). The organic layers were combined, washed with satd. aq. NaCl, dried over anh. Na₂SO₄, filtered, and concentrated in vacuo. Purification by silica gel column chromatography (up to 30% EtOAc in hexanes) provided the title compound as a white solid (1.40 g, 4.69 mmol, 73%)

¹H NMR (600 MHz, CDCl₃) δ 7.30 (d, J=7.0 Hz, 2H), 5.01 (s, 1H), 4.31-4.24 (m, 4H), 1.29 (t, J=7.4 Hz, 6H) ppm.

¹³C NMR (150 MHz, CDCl₃) δ 165.54, 161.02 (dd, J=254.0, 7.8 Hz), 116.78 (t, J=18.5 Hz), 116.20 (t, J=3.4 Hz), 115.94-115.48 (m), 113.88 (t, J=12.4 Hz), 62.60, 47.24, 13.87 ppm.

IR: 2856, 2178, 1695 cm⁻¹

LCMS: [M+H]⁺ 298.

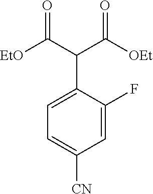 [SMILES: CCOC(=O)C(C(=O)OCC)c1ccc(C#N)cc1F]

(93)

### Diethyl 2-(4-cyano-2-fluorophenyl)malonate

To a dry flask, under nitrogen, was added 3,4-difluorobenzonitrile (4.00 g, 28.8 mmol, 1 equiv), potassium carbonate (7.95 g, 57.5 mmol, 2.00 equiv), diethyl malonate (4.66 g, 29.1 mmol, 1.01 equiv), and anhydrous DMF (24 ml) and was heated at 65° C. until starting material was consumed based on TLC. The reaction mixture was cooled to r.t. and added to a separatory funnel containing 50 ml of 1 N HCl. The mixture extracted with EtOAc (×3), and the combined organic layers were washed with water and satd. aq. NaCl. The organic layer was dried over anh. Na₂SO₄, filtered, and concentrated in vacuo. Purification by silica gel column chromatography (up to 30% EtOAc in hexanes) Provided the title compound as a colorless oil (6.73 g, 24.1 mmol, 84%)

¹H NMR (600 MHz, CDCl₃) δ 7.64 (t, J=7.6 Hz, 1H), 7.47 (dd, J=8.0, 1.6 Hz, 1H), 7.38 (dd, J=9.2, 1.6 Hz, 1H), 4.98 (s, 1H), 4.28-4.18 (m, 4H), 1.26 (t, J=7.2 Hz, 6H).

¹³C NMR (150 MHz, CDCl₃) δ 166.55, 160.04 (d, J=252.0 Hz), 131.98 (d, J=3.5 Hz), 128.26 (d, J=4.0 Hz), 126.21 (d, J=14.4 Hz), 119.23 (d, J=26.0 Hz), 117.28 (d, J=2.9 Hz), 113.71 (d, J=9.8 Hz), 62.55, 50.44 (d, J=2.9 Hz), 14.00.

IR: 2984, 2236, 1733, 1219 cm⁻¹

LCMS: [M+H]⁺ 280

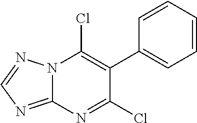 [SMILES: Clc1nc2ncnn2c(Cl)c1-c1ccccc1]

(96)

### 5,7-dichloro-6-phenyl-[1,2,4]triazolo[1,5-a]pyrimidine

Following General Procedure B using diethyl 2-phenylmalonate (780 mg, 3.30 mmol) and 3-amino-1,2,4-triazole (292 mg, 3.47 mmol). Then using the intermediate sodium 6-phenyl-[1,2,4]triazolo[1,5-a]pyrimidine-5,7-bis(olate) (740 mg, 2.71 mmol) and _{POCl3} (7.41 g, 48.4 mmol) provide the title compound as a brown solid (527 mg, 1.99 mmol, 73%).

¹H NMR (500 MHz, CDCl₃) δ 8.56 (s, 1H), 7.58-7.51 (m, 3H), 7.38-7.34 (m, 2H).

¹³C NMR (126 MHz, CDCl₃) δ 156.93, 156.65, 153.35, 139.90, 131.72, 130.07, 129.95, 129.16, 124.01.

IR (KBr) ν 3437, 1637, 1458, 1383, 1267, 1205, 1180, 867, 802, 764, 740, 698, 652 cm⁻¹

HRMS (ES+) calculated for C₁₁H₇Cl₂N₄ [M+H]⁺ 265.0042, found 265.0043.

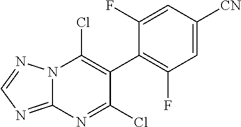 [SMILES: N#Cc1cc(F)c(-c2c(Cl)nc3ncnn3c2Cl)c(F)c1]

(109)

### 4-(5,7-Dichloro-[1,2,4]triazolo[1,5-a]pyrimidin-6-yl)-3,5-difluorobenzonitrile (22)

Following General Procedure B using diethyl 2-(4-cyano-2,6-difluorophenyl)malonate (500 mg, 1.68 mmol) and 3-amino-1,2,4-triazole (148 mg, 1.77 mmol). Intermediate sodium 6-(4-cyano-2,6-difluorophenyl)-[1,2,4]triazolo[1,5-a]pyrimidine-5,7-bis(olate) (400 mg, 1.20 mmol) and POCl₃ (3.28 g, 21.4 mmol) provide the title compound as a brown solid (210 mg, 1.20 mmol, 53%).

¹H NMR (600 MHz, CDCl₃) δ 8.65 (s, 1H), 7.47 (d, J=6.2 Hz, 2H) ppm.

¹³C NMR (150 MHz, CDCl₃) δ 161.21 (d, J=6.4 Hz), 159.51 (d, J=6.2 Hz), 157.66, 155.57, 116.85, 116.62-116.30 (m), 115.86 (t, J=3.5 Hz), 114.64 (t, J=19.6 Hz) ppm.

IR: 3087, 2195, 1571, 1338, 1195 cm⁻¹

LCMS: [M+H]⁺ 327

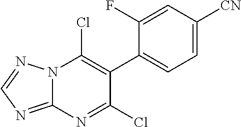 [SMILES: N#Cc1ccc(-c2c(Cl)nc3ncnn3c2Cl)c(F)c1]

(110)

### 4-(5,7-Dichloro-[1,2,4]triazolo[1,5-a]pyrimidin-6-yl)-3-fluorobenzonitrile (23)

Following General Procedure B using diethyl 2-(4-cyano-2,6-difluorophenyl)malonate (500 mg, 1.68 mmol) and 3-amino-1,2,4-triazole (148 mg, 1.77 mmol). Then using the intermediate sodium 6-(4-cyano-2-fluorophenyl)-[1,2,4]triazolo[1,5-a]pyrimidine-5,7-bis(olate) (3.00 g, 9.52 mmol) and POCl₃ (26.0 g, 169 mmol to provide the title compound as a brown solid (1.97 g, 9.52 mmol, 67%).

¹H NMR (600 MHz, CDCl₃) δ 8.61 (s, 1H), 7.68 (dd, J=7.9, 1.5 Hz, 1H), 7.61 (dd, J=8.6, 1.5 Hz, 1H), 7.54 (t, J=7.4 Hz, 1H) ppm.

¹³C NMR (150 MHz, CDCl₃) δ 160.47, 158.79, 157.48, 155.52, 153.79, 140.73, 133.32, 128.86 (d, J=4.6 Hz), 124.68 (d, J=15.7 Hz), 120.42 (d, J=24.9 Hz), 116.76 (d, J=2.9 Hz), 116.69, 116.36 (d, J=9.3 Hz) ppm.

### Example 3—Microtubule Stabilization Assay

All test compounds were evaluated in a previously described cell-based assay of MT-stabilization (see Kovalevich, J., et al. (2016) Characterization of Brain-Penetrant Pyrimidine-Containing Molecules with Differential Microtubule-Stabilizing Activities Developed as Potential Therapeutic Agents for Alzheimer's Disease and Related Tauopathies. J Pharmacol Exp Ther 357, 432-450, incorporated herein by reference), in which compound-dependent changes in tubulin polymerization and total tubulin levels were determined after 4 h of incubation of QBI293 cells with 1 and 10 μM compound via quantification of acetylated α-tubulin and total tubulin in cell lysates by ELISA.

The observation that specific structural features within the triazolopyrimidines are critical to elicit an increase in MT stability and mass led to a more systematic exploration of the SAR of the triazolopyrimidine scaffold, thereby elucidating the MT-stabilizing SAR of triazolopyrimidines modified at C6 and C7. The results from these studies led to the identification of selected congeners with improved in vitro activity and physicochemical properties compared to the existing lead. In particular, these studies have taught that the desired Class I MT-stabilizing effects of triazolopyrimidines generally require the presence of an electron-poor phenyl ring at C6 (R₆ in Formula I). Different fluorinated phenyl fragments can be tolerated leading to active Class I congeners, especially those bearing either one or two fluorine atoms in the ortho position, and a para position that is either unsubstituted or substituted with an additional fluorine or other electron-withdrawing groups, such as nitro and nitrile. Moreover, the nature of the fragment linked at C7 can play an important role in determining both potency as well as cellular phenotype elicited by MT-active triazolopyrimidines, and that the presence of a substituted nitrogen at this position may be a necessary condition for Class I activity. Finally, an evaluation of the effect of the chiral configuration of the branched amine fragment at C7 was also conducted. In the majority of cases examined the chirality of the amine was found to influence the potency of the compound with one enantiomer being typically more active than the other.

Novel C6 nitrile- and alkyne-substituted triazolopyrimidine examples that have improved in vitro activity relative to previously described examples, with the general structure depicted in Formula I. The activity of the novel C6-nitrile substituted compounds were tested in a previously described cellular assay of MT-stabilization (Kovalevich, J., et al. (2016)). As summarized in Table 2. below, eighteen examples were synthesized and tested. In all cases, these compounds were confirmed to be active Class I triazolopyrimidines. In addition, among this series of congeners, selected compounds, such as 64, 66, 69, 70 and 75-77, were identified with improved in vitro potency compared to lead compound 3. Table 2 provides fold-changes in acetylated α-tubulin and total α-tubulin levels in QB1293 cells after 4 h incubation with test compounds at either 1 or 10 μM. Data are expressed as the fold-change relative to vehicle-treated cells. *p<0.05 and **p<0.01 by one-way ANOVA.

TABLE 2

| | | AcTub | | α-Tub | | |
|---|---|---|---|---|---|---|
| CNDR # | Structure | l μM | 10 μM | 1 μM | 10 μM | Class |
| 51967 (19) | 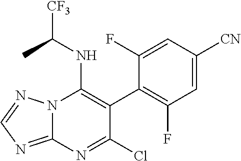 [SMILES: C[C@H](Nc1c(-c2c(F)cc(C#N)cc2F)c(Cl)nc2ncnn12)C(F)(F)F] | 1.90* ± 0.06 | 3.56** ± 0.31 | 0.94 ± 0.02 | 0.95 ± 0.05 | 1 |
| 51989 (61) | 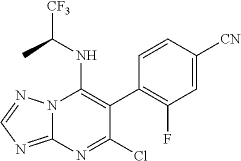 [SMILES: C[C@H](Nc1c(-c2ccc(C#N)cc2F)c(Cl)nc2ncnn12)C(F)(F)F] | 2.01* ± 0.38 | 3.64** +/− 0.26 | 1.00 +/− 0.01 | 1.00 +/− 0.01 | 1 |
| 51998 (62) | 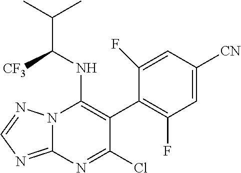 [SMILES: CC(C)[C@@H](Nc1c(-c2c(F)cc(C#N)cc2F)c(Cl)nc2ncnn12)C(F)(F)F] | 0.99 +/- 0.06 | 2.21** +/− 0.13 | 1.02 +/− 0.01 | 1.01 +/− 0.02 | 1 |
| 51995 (63) | 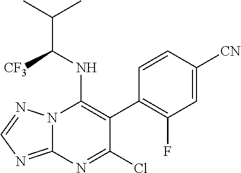 [SMILES: CC(C)[C@@H](Nc1c(-c2ccc(C#N)cc2F)c(Cl)nc2ncnn12)C(F)(F)F] | 1.76** ± 0.06 | 2.72** +/− 0.17 | 0.99 +/− 0.01 | 0.98 +/− 0.01 | 1 |
| 51992 (66) | 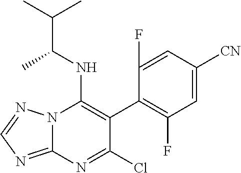 [SMILES: CC(C)[C@@H](C)Nc1c(-c2c(F)cc(C#N)cc2F)c(Cl)nc2ncnn12] | 3.80* +/− 0.15 | 4.28** +/− 0.14 | 0.93 +/− 0.02 | 0.94 +/− 0.02 | 1 |
| 52017 (67) | 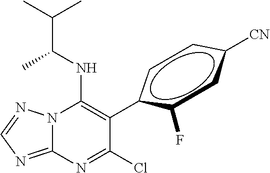 | 2.79 +/− 1.11 | 3.42* +/− 0.55 | 0.99 +/− 0.02 | 0.93 +/− 0.02 | 1 |
| 52016 (68) | 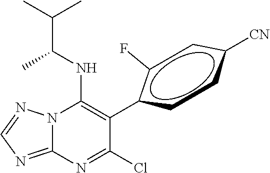 | 2.15* +/− 0.36 | 2.29* +/− 0.30 | 0.95 +/− 0.02 | 0.92 +/− 0.01 | 1 |
| 51997 (69) | 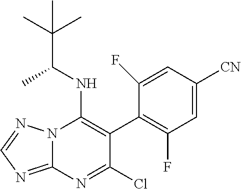 [SMILES: C[C@@H](Nc1c(-c2c(F)cc(C#N)cc2F)c(Cl)nc2ncnn12)C(C)(C)C] | 2.83 ± 0.33 | 4.60 ± 0.35 | 0.98 +/− 0.03 | 1.03 +/− 0.03 | 1 |
| 51990 (70) | 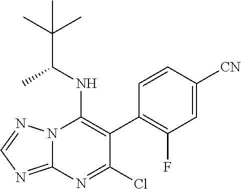 [SMILES: C[C@@H](Nc1c(-c2ccc(C#N)cc2F)c(Cl)nc2ncnn12)C(C)(C)C] | 4.41* ± 0.27 | 7.67 ± 0.70 | 0.86 +/− 0.03 | 0.93 +/− 0.08 | 1 |
| 52005 (73) | 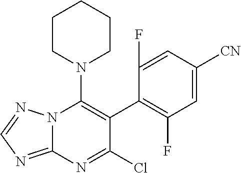 [SMILES: N#Cc1cc(F)c(-c2c(Cl)nc3ncnn3c2N2CCCCC2)c(F)c1] | 1.00 +/− 0.02 | 2.19** +/− 0.23 | 1.00 +/− 0.02 | 1.07 +/− 0.01 | 1 |
| 52015 (74) | 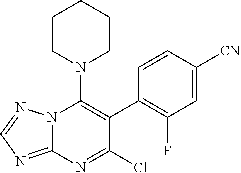 [SMILES: N#Cc1ccc(-c2c(Cl)nc3ncnn3c2N2CCCCC2)c(F)c1] | 2.25* +/− 0.51 | 3.20** +/− 0.33 | 0.98 +/− 0.02 | 0.95 +/− 0.03 | 1 |
| 51991 (75) | 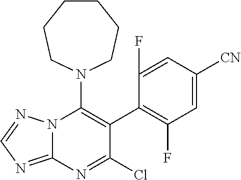 [SMILES: N#Cc1cc(F)c(-c2c(Cl)nc3ncnn3c2N2CCCCCC2)c(F)c1] | 6.40** ± 0.61 | 11.2** +/− 0.77 | 0.98 +/- 0.03 | 0.99 +/− 0.02 | 1 |
| 51996 (76) | 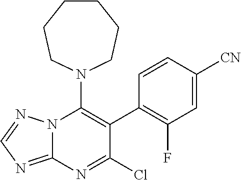 [SMILES: N#Cc1ccc(-c2c(Cl)nc3ncnn3c2N2CCCCCC2)c(F)c1] | 7.17** ± 1.09 | 12.6** +/− 2.8 | 1.04 +/− 0.05 | 0.99 +/− 0.03 | 1 |
| 52007 (77) | 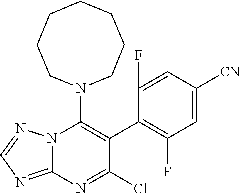 [SMILES: N#Cc1cc(F)c(-c2c(Cl)nc3ncnn3c2N2CCCCCCC2)c(F)c1] | 3.24** +/− 0.80 | 4.40** +/− 0.34 | 1.01 +/− 0.02 | 0.99 +/− 0.02 | 1 |
| 52004 (78) | 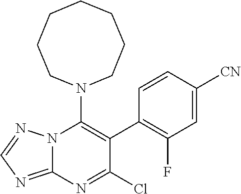 [SMILES: N#Cc1ccc(-c2c(Cl)nc3ncnn3c2N2CCCCCCC2)c(F)c1] | 1.09 +/− 0.22 | 2.89** +/− 0.56 | 0.97 +/− 0.02 | 0.95 +/− 0.07 | 1 |

These results demonstrated that the tested C6 nitrile-substituted triazolopyrimidine compounds according to the present disclosure have improved in vitro activity relative to previously described examples.

### Example 4—Plasma and Brain Compound Determinations

CD1 mice (n=3) were dosed concurrently with CNDR-51990 and CNDR-51993 (representing a 1:1 mixture of two diastereoisomers, 52017 (67) and 52016 (68)) at 2.5 mg/kg each). One hour after dosing, mice were euthanized and blood collected, followed by perfusion with saline. Mouse brains were homogenized in 10 mM ammonium acetate, pH 5.7 (1:2; w/v) using a handheld sonic homogenizer. Mouse plasma was obtained from blood that was collected into a 1.5 ml tube containing 0.5M EDTA solution and which was centrifuged for 10 minutes at 4500 g at 4° C. Aliquots (50 μl) of brain homogenates or plasma were mixed with 0.2 ml of acetonitrile, centrifuged at 15,000×g, and the resulting supernatant was used for subsequent LC-MS/MS analysis. The LC-MS/MS analysis was conducted by Inotiv, Inc., utilizing methods essentially as previously described (Lou et al., J. Med. Chem. 57:6116-27, 2014). Results are provided in Table 3, below.

TABLE 3

| | | Plasma | Brain | |
|---|---|---|---|---|
| Compound | | Concentration | Concentration | |
| Number | Animal ID | (ng/mL) | (ng/g) | Brain/Plasma |
| 51990 | 1 | 175 | 158 | 0.90 |
| (70) | 2 | 133 | 186 | 1.40 |
| | 3 | 146 | 206 | 1.41 |
| | Mean | 151 | 183 | 1.21 |
| | SD | 22 | 24 | 0.29 |
| | n | 3 | 3 | 3 |
| 51993 | 1 | 242 | 209 | 0.86 |
| (1:1 | 2 | 186 | 211 | 1.13 |
| mixture of | 3 | 190 | 254 | 1.34 |
| 52017 (67) | Mean | 206 | 225 | 1.09 |
| and 52016 | SD | 31 | 25 | 0.24 |
| (68)) | n | 3 | 3 | 3 |

### Example 6—Additional Microtubule Stabilizing Compounds

Additional microtubule stabilizing compounds according to the present disclosure were prepare in accordance with Schemes 1-4, below.

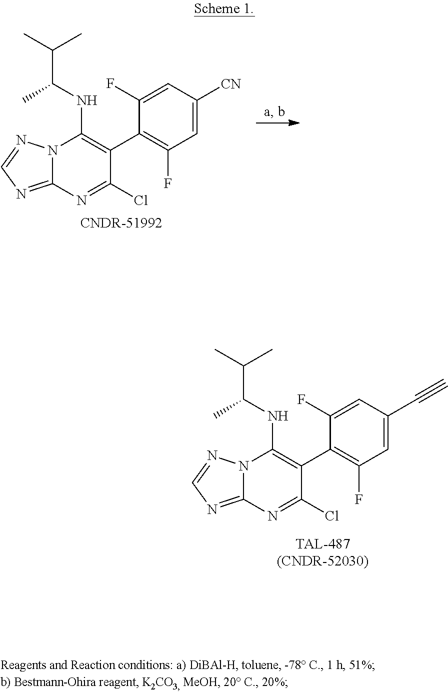

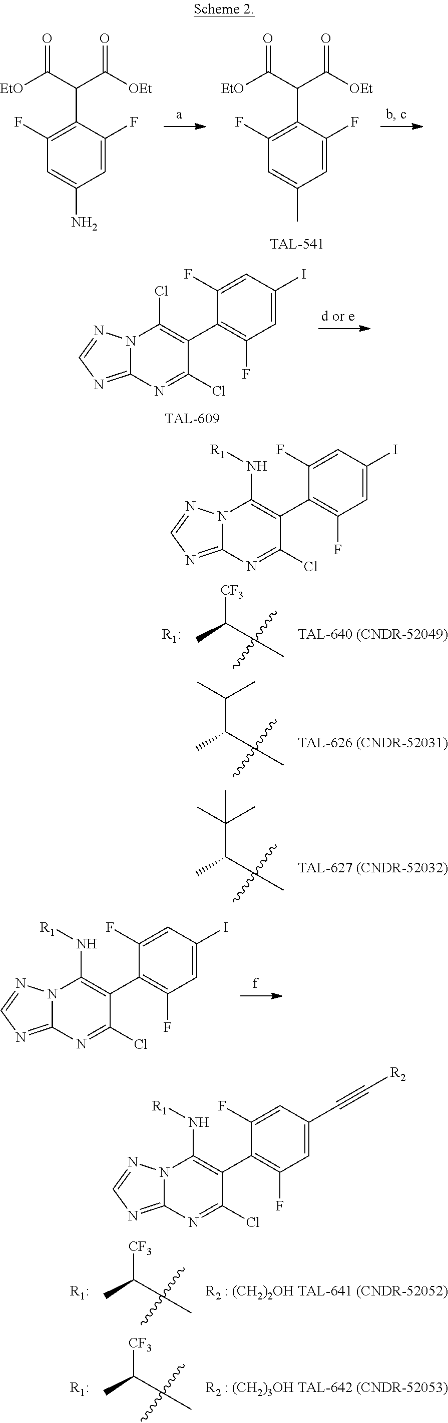

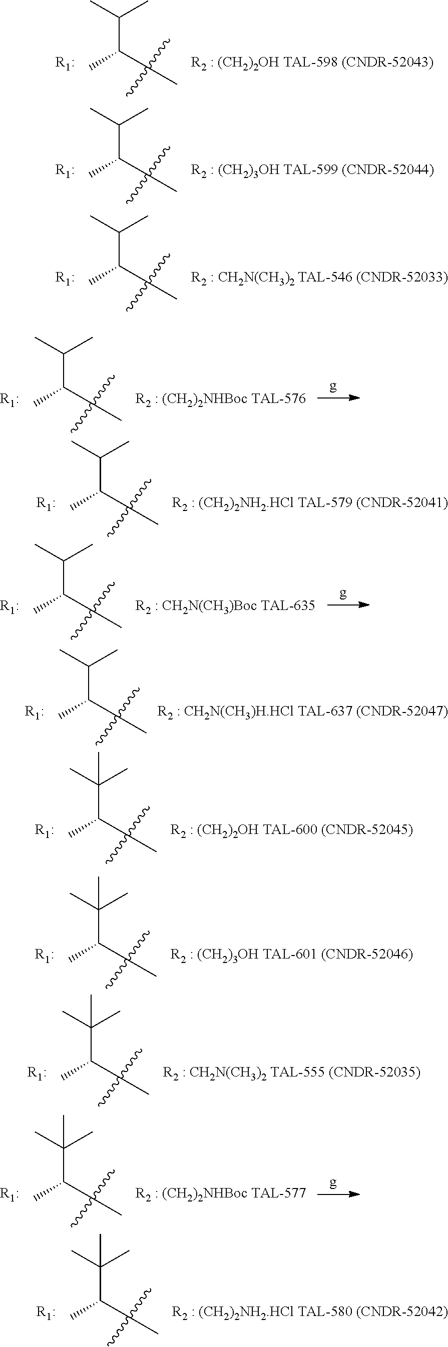

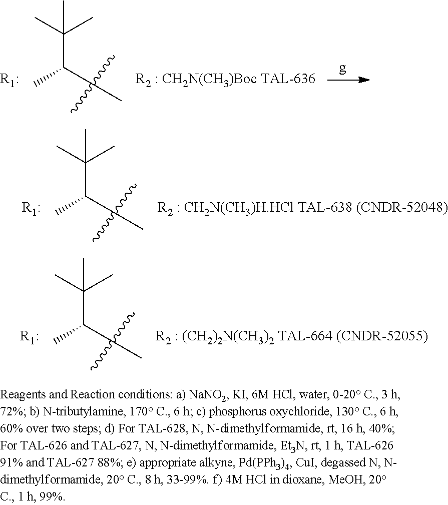

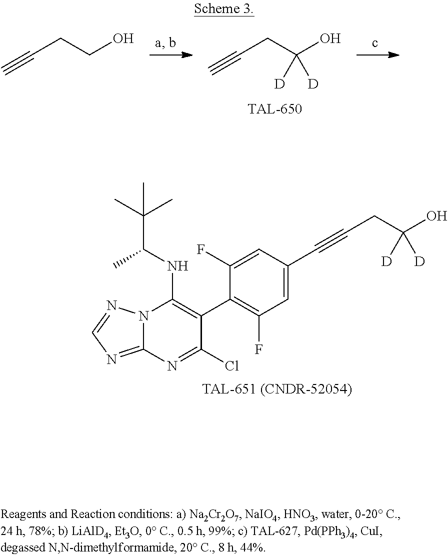

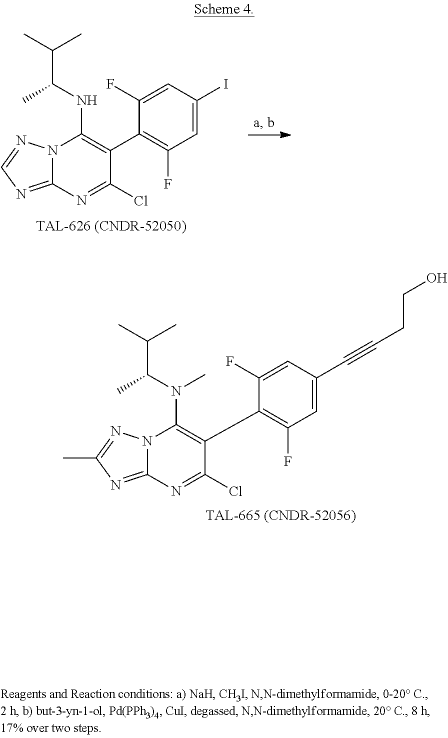 [SMILES: CC(C)[C@@H](C)Nc1c(-c2c(F)cc(I)cc2F)c(Cl)nc2ncnn12 ; Cc1nc2nc(Cl)c(-c3c(F)cc(C#CCCO)cc3F)c(N(C)[C@H](C)C(C)C)n2n1]

Compounds produced according to the preceding synthetic schemes were for microtubule stabilization. Table 4, below, describes the MT-stabilizing activity of triazolopyrimidines. Fold-changes in acetylated α-tubulin (AcTub) and α-tubulin (α-Tub) levels in QB1293 cells after 4 h incubation with test compounds at either 1 or 10 μM. Reported values for AcTub and α-Tub represent the fold-change over control (DMSO)-treated cells (*p<0.05 and **p<0.01 by one-way ANOVA); numbers in parentheses represent the fold-change of AcTub over positive control-treated cells (i.e., 100 nM of cevipabulin). Changes of +/−15% of control are deemed to be within the day-to-day assay variability and non-significant.

TABLE 4

| | | AcTub | | α-Tub | | |
|---|---|---|---|---|---|---|
| Cpd # | Structure | 1 μM | 10 μM | l μM | 10 μM | B/P |
| TAL- 640 CNDR- 052049 | 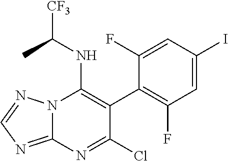 [SMILES: C[C@H](Nc1c(-c2c(F)cc(I)cc2F)c(Cl)nc2ncnn12)C(F)(F)F] | 1.90 +/− 0.04 (0.25) | 6.90** +/− 0.68 (0.93) | 1.01 +/− 0.04 | 0.93 +/− 0.02 | |
| TAL- 626 CNDR- 052031 | 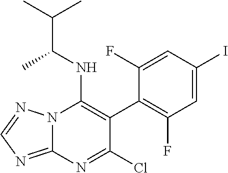 [SMILES: CC(C)[C@@H](C)Nc1c(-c2c(F)cc(I)cc2F)c(Cl)nc2ncnn12] | 0.74 +/− 0.10 (0.13) | 2.88* +/− 0.07 (0.53) | 0.74 +/− 0.31 | 0.93 +/− 0.01 | |
| TAL- 627 CNDR- 052032 | 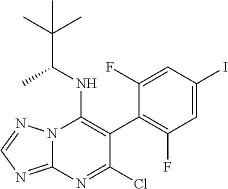 [SMILES: C[C@@H](Nc1c(-c2c(F)cc(I)cc2F)c(Cl)nc2ncnn12)C(C)(C)C] | 2.78 +/− 0.07 (0.51) | 6.94** +/− 1.14 (1.27) | 0.97 +/− 0.02 | 0.89+/− 0.03 | |
| TAL- 487 CNDR- 052030 | 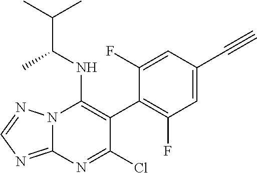 [SMILES: C#Cc1cc(F)c(-c2c(Cl)nc3ncnn3c2N[C@H](C)C(C)C)c(F)c1] | 2.59** +/− 0.19 (0.28) | 5.79** +/− 0.18 (0.63) | 0.98 +/− 0.01 | 1.03 +/− 0.01 | |
| TAL- 641 CNDR- 052052 | 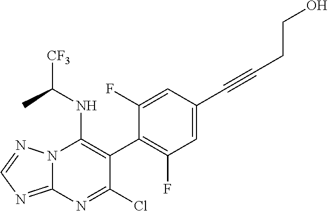 [SMILES: C[C@H](Nc1c(-c2c(F)cc(C#CCCO)cc2F)c(Cl)nc2ncnn12)C(F)(F)F] | 3.68** +/− 0.29 (0.49) | 16.1** +/− 1.19 (2.16) | 0.98 +/− 0.03 | 0.83** +/− 0.01 | |
| TAL- 642 CNDR- 052053 | 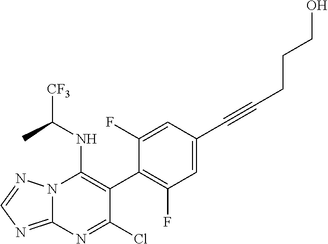 [SMILES: C[C@H](Nc1c(-c2c(F)cc(C#CCCCO)cc2F)c(Cl)nc2ncnn12)C(F)(F)F] | 17.1** +/− 0.48 (2.29) | 14.4** +/− 0.57 (1.93) | 0.93 +/− 0.02 | 0.87 +/− 0.03 | |
| TAL- 598 CNDR- 052043 | 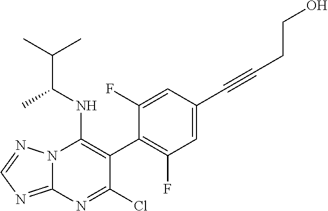 [SMILES: CC(C)[C@@H](C)Nc1c(-c2c(F)cc(C#CCCO)cc2F)c(Cl)nc2ncnn12] | 4.24** +/− 0.20 (0.66) | 5.60** +/− 0.78 (0.87) | 0.92 +/− 0.02 | 0.80* +/− 0.05 | 0.44 |
| TAL- 599 CNDR- 052044 | 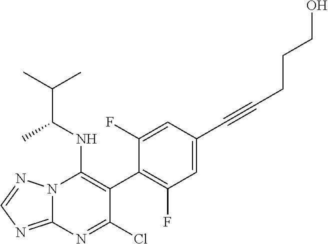 [SMILES: CC(C)[C@@H](C)Nc1c(-c2c(F)cc(C#CCCCO)cc2F)c(Cl)nc2ncnn12] | 2.60 +/− 0.16 (0.40) | 4.43** +/− 0.56 (0.69) | 1.00 +/− 0.08 | 1.37 +/− 0.05 | |
| TAL- 546 CNDR- 052033 | 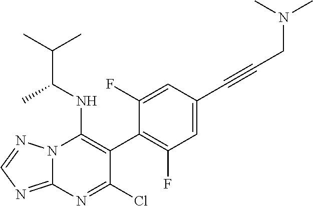 [SMILES: CC(C)[C@@H](C)Nc1c(-c2c(F)cc(C#CCN(C)C)cc2F)c(Cl)nc2ncnn12] | 2.21 +/− 0.60 (0.40) | 7.68** +/− 0.85 (1.40) | 0.98 +/− 0.04 | 1.03 +/− 0.04 | |
| TAL- 579 CNDR- 052041 | 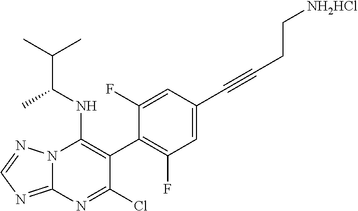 | 2.30** +/− 0.31 (0.76) | 2.58** +/− 0.23 (0.86) | 0.99 +/− 0.05 | 1.00 +/− 0.03 | |
| TAL- 637 CNDR- 052047 | 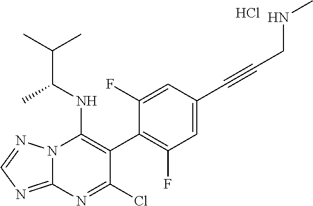 [SMILES: CNCC#Cc1cc(F)c(-c2c(Cl)nc3ncnn3c2N[C@H](C)C(C)C)c(F)c1 ; Cl] | 1.98** +/− 0.09 (0.26) | 5.92** +/− 0.14 (0.79) | 0.96 +/− 0.01 | 0.96 +/− 0.03 | |
| TAL- 600 CNDR- 052045 | 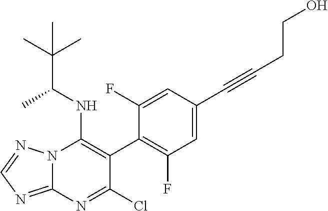 [SMILES: C[C@@H](Nc1c(-c2c(F)cc(C#CCCO)cc2F)c(Cl)nc2ncnn12)C(C)(C)C] | 3.67* +/− 0.34 (0.57) | 5.25** +/− 0.89 (0.82) | 0.98 +/− 0.04 | 0.98 +/− 0.20 | 0.47 |
| TAL- 601 CNDR- 052046 | 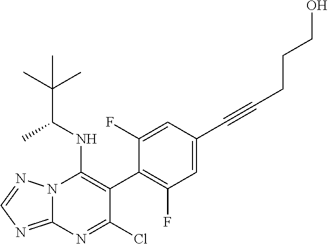 [SMILES: C[C@@H](Nc1c(-c2c(F)cc(C#CCCCO)cc2F)c(Cl)nc2ncnn12)C(C)(C)C] | 4.78** +/− 1.02 (0.74) | 5.59** +/− 0.26 (0.87) | 1.18 +/− 0.11 | 1.14 +/− 0.08 | |
| TAL- 555 CNDR- 052035 | 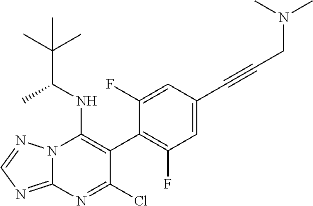 [SMILES: C[C@@H](Nc1c(-c2c(F)cc(C#CCN(C)C)cc2F)c(Cl)nc2ncnn12)C(C)(C)C] | 1.36 +/− 0.08 (0.45) | 4.32** +/− 0.28 (1.43) | 1.00 +/− 0.05 | 0.84+/− 0.01 | |
| TAL- 580 CNDR- 052042 | 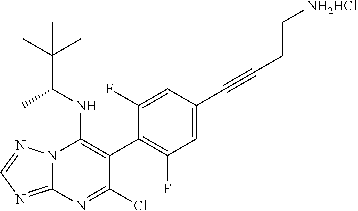 | 2.49** +/− 0.17 (0.83) | 3.65** +/− 0.58 (1.21) | 0.96 +/− 0.03 | 0.91 +/− 0.12 | 1.04 |
| TAL- 638 CNDR- 052048 | 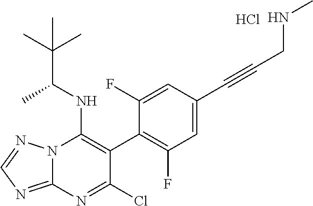 [SMILES: CNCC#Cc1cc(F)c(-c2c(Cl)nc3ncnn3c2N[C@H](C)C(C)(C)C)c(F)c1 ; Cl] | 6.03** +/− 0.64 (0.81) | 8.67** +/− 0.16 (1.16) | 0.93 +/− 0.02 | 1.00 +/− 0.03 | |
| TAL- 651 CNDR- 052054 | 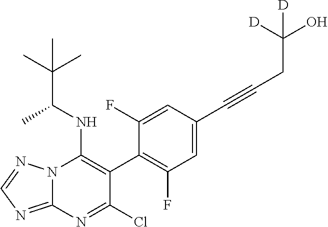 [SMILES: [2H]C([2H])(O)CC#Cc1cc(F)c(-c2c(Cl)nc3ncnn3c2N[C@H](C)C(C)(C)C)c(F)c1] | 12.6** +/− 0.30 (1.69) | 16.3** +/− 0.19 (2.18) | 1.02 +/− 0.02 | 1.01 +/− 0.02 | 0.46 |
| TAL- 664 CNDR- 052055 | 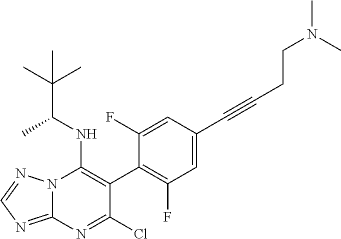 [SMILES: C[C@@H](Nc1c(-c2c(F)cc(C#CCCN(C)C)cc2F)c(Cl)nc2ncnn12)C(C)(C)C] | 10.0** +/− 0.47 (1.18) | 8.56** +/− 0.56 (1.00) | 0.88 +/− 0.02 | 0.96 +/− 0.01 | |
| TAL- 665 CNDR- 052056 | 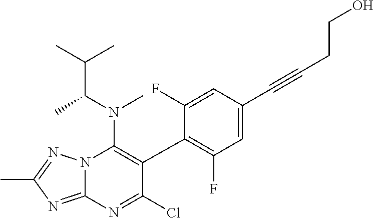 [SMILES: Cc1nc2nc(Cl)c(-c3c(F)cc(C#CCCO)cc3F)c(N(C)[C@H](C)C(C)C)n2n1] | 1.05 +/− 0.14 (0.12) | 2.85** +/− 0.21 (0.33) | 0.91 +/− 0.03 | 0.96 +/− 0.02 | |

Materials and Methods. All solvents were of reagent grade. All reagents were purchased from Aldrich or Enamine and used as received. Thin-layer chromatography (TLC) was performed with 0.25 mm E. Merck precoated silica gel plates. Silica gel column chromatography was performed with silica gel 60 (particle size 0.040-0.062 mm) supplied by Silicycle and Sorbent Technologies. TLC spots were detected by viewing under a UV light. Proton (¹H) and carbon (¹³C) NMR spectra were recorded on a 600 MHz Bruker AVANCE III spectrometer. Chemical shifts were reported relative to solvents. Data for 1H NMR spectra are reported as follows: chemical shift [ppm, referenced to protium; s=singlet, d=doublet, t=triplet, q=quartet, quint=quintet, dd=doublet of doublets, bs=broad singlet, mn=multiplet, coupling constant (Hz), and integration]. High-resolution mass spectra were measured using an Agilent 6230 time-of-flight mass spectrometer with a Jet stream electrospray ionization source. HPLC was performed with a Gilson HPLC equipped with UV and a mass detector. All samples were analyzed employing a linear gradient from 10 to 90% of ACN in H₂O over 8 min and flow rate of 1 mL/min. Preparative reverse-phase HPLC purifications were performed on a Gilson instrument employing Waters SunFire preparative Cis OBD columns (5 μm 19 mm Å˜50 mm or 19 mm Å˜100 mm). All final compounds were found to be >95% pure by HPLC.

General procedure A. To a solution of Iodo-triazolopyrimidine (1 equiv) in degassed DMF (0.2 M), were added successively CuI (0.15 equiv), triethylamine (3 equiv) and the desired alkyne (3 equiv). The mixture was degassed and backfilled with Nitrogen before Tetrakis palladium (0.1 equiv) was added. The mixture was degassed and backfilled with Nitrogen three times and the mixture was stirred at rt for 8 hours before it was quenched with water and extracted with EtOAc twice. The combined organic fractions were washed with brine, dried over MgSO₄ and concentrated under reduced pressure. Purification via silica gel column chromatography (Hexanes/EtOAc) or by reverse phase HPLC (water/ACN+0.1% FA, (10 to 90%), 20 mL/min, 20 min ramp time) to furnish the desired compound.

General procedure B. To a solution of Boc-protected alkynylated TPD (1 equiv) in MeOH (0.1 M) was added a 4 M solution of HCl in dioxane (18 equiv). After 3 hours at rt, the mixture was evaporated under reduced pressure to furnish the title compound as a HCl salt as a yellow or brown solid.

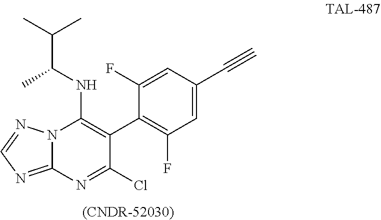

(R)-5-chloro-6-(4-ethynyl-2,6-difluorophenyl)-N-(3-methylbutan-2-yl)-[1,2,4]triazolo[1,5-a]pyrimidin-7-amine. To a solution of (R)-4-(5-chloro-7-((3-methylbutan-2-yl)amino)-[1,2,4]triazolo[1,5-a]pyrimidin-6-yl)-3,5-difluorobenzonitrile (0.047 g, 0.125 mmol, 1 equiv) in dry toluene (12 mL) at 0° C., was added DiBAl-H (0.170 mL, 1.1 M, 0.027 g, 0.182 mmol, 1.5 equiv) dropwise and the mixture was stirred for 1 hour at this temperature before it was quenched with a 1 M solution of HCl. The aqueous layer was extracted twice with EtOAc, then the combined organic fractions were washed with brine, dried over MgSO₄ and concentrated under reduced pressure to furnish the aldehyde intermediate which was directly dissolved into MeOH (4 mL). K₂CO₃ (0.025 g, 0.182 mmol, 1.5 equiv) and dimethyl (1-diazo-2-oxopropyl)phosphonate (0.035 g, 0.182 mmol, 1.5 equiv) were successively added and the mixture was stirred at rt for 3 hours. The reaction was then filtered over sintered glass and the filtrate was evaporated under reduced pressure. Purification via silica gel column chromatography (Hexanes/EtOAc: 90/10 to 75/25) to furnish the title compound as an off-white solid (0.005 g, 0.012 mmol, 10% over two steps). ¹H NMR (600 MHz, CDCl₃) δ 8.39 (s, 1H), 7.25 (d, J=7.5 Hz, 2H), 6.38 (bs, 1H), 3.30 (bs, 1H), 2.71 (d, J=5.4 Hz, 1H), 1.72-1.67 (m, 1H), 1.11 (d, J=6.6 Hz, 3H), 0.86 (d, J=6.6 Hz, 3H), 0.84 (d, J=6.6 Hz, 3H) ppm. HRMS (ES+) calculated for C₁₈H₁₇N₅F₂Cl [M+H]⁺: 376.1135, found 376.1139.

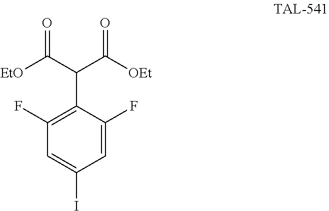 [SMILES: CCOC(=O)C(C(=O)OCC)c1c(F)cc(I)cc1F]

diethyl 2-(2,6-difluoro-4-iodophenyl)malonate. To a solution of diethyl 2-(4-amino-2,6-difluorophenyl)malonate (2.050 g, 7.136 mmol, 1 equiv) in 6 N HCl (12 mL, 71.36 mmol, 10 equiv) cooled to 0° C., a solution of NaNO₂ (0.492 g, 7.136 mmol, 1 equiv) in water (2.7 mL) was added dropwise. The resulting solution was added dropwise to a solution of KI (4.916 g, 29.62 mmol, 4.15 equiv) in water (5 mL) keeping the temperature at 0° C. The reaction mixture was allowed to warm to room temperature and stirred for 3 h, then it was stopped and extracted twice with EtOAc. The combined layers were washed in sequence with 10% Na₂S203 and brine, then dried over MgSO₄ and concentrated under reduced pressure. Purification via silica gel column chromatography (Hexanes/EtOAc: 100/0 to 75/25) to furnish the title compound (2.054 g, 5.159 mmol, 72%) as a yellow oil. ¹H NMR (600 MHz, CDCl₃) δ 7.31 (d, J=7.0 Hz, 1H), 4.89 (s, 1H), 4.25 (q, J=7.1 Hz, 4H), 1.27 (t, J=7.2 Hz, 6H) ppm. ¹³C NMR (151 MHz, CDCl₃) δ 166.36, 160.77 (dd, J=255.2, 7.8 Hz), 121.41 (dd, J=23.6, 4.8 Hz), 101.61 (dt, J=20.3, 14.7 Hz), 62.45, 47.22, 14.05 ppm. LCMS: [M+H]⁺: 339.

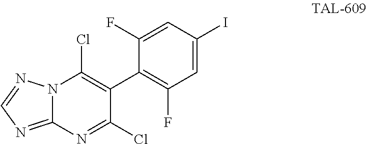 [SMILES: Fc1cc(I)cc(F)c1-c1c(Cl)nc2ncnn2c1Cl]

5,7-dichloro-6-(2,6-difluoro-4-iodophenyl)-[1,2,4]triazolo[1,5-a]pyrimidine. In a pressure flask flushed with Nitrogen, were charged TAL-541 (1.920 g, 6.459 mmol, 1 equiv), 1H-1,2,4-triazol-5-amine (0.570 g, 6.782 mmol, 1.05 equiv) and tributylamine (1.620 mL, 6.782 mmol, 1.05 equiv). The mixture was heated to 170° C. for 3 hours. 10 mL of toluene were then added at 110° C. followed by a 50% NaOH solution (1.023 mL, 19.38 mmol, 3 equiv) at 50° C. Once at rt, the mixture was filtered over sintered glass and rinsed twice with toluene to furnish the bis-phenolate (2.068 g, 6.621 mmol, 97%) that is engaged in the next step without further purification. In a round bottom flask charged with the intermediate bis-phenolate (2.000 g, 4.608 mmol, 1 equiv) was added phosphoryl chloride (7.670 mL, 82.020 mmol, 17.8 equiv) and the mixture was heated to 130° C. for 6 hours. The reaction was poured over ice, the aqueous phase was extracted twice with CH₂Cl₂ and the combined organic fractions were washed with brine, dried over MgSO₄ and concentrated under reduced pressure. Purification via silica gel column chromatography (Hexanes/EtOAc; 100/0 to 70/30) to furnish the title compound as an off-white solid (1.520 g, 3.56 mmol, 77%). ¹H NMR (600 MHz, CDCl₃) δ 8.61 (s, 1H), 7.52 (d, J=6.4 Hz, 2H) ppm. ¹³C NMR (151 MHz, CDCl₃) δ 160.45 (d, J=6.2 Hz), 158.75 (d, J=6.2 Hz), 157.28, 156.13, 153.85, 141.28, 122.05 (dd, J=23.0, 4.2 Hz), 112.35, 109.18 (t, J=19.8 Hz), 95.68 (t, J=10.2 Hz) ppm. HRMS (ES+) calculated for C₁₁H₄N₄F₂ICl₂ [M+H]⁺: 426.8820, found 426.8812.

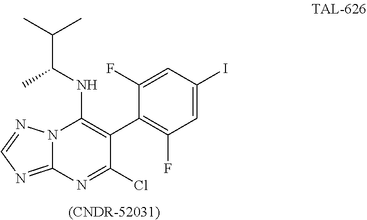

(R)-5-chloro-6-(2,6-difluoro-4-iodophenyl)-N-(3-methylbutan-2-yl)-[1,2,4]triazolo[1,5-a]pyrimidin-7-amine. To a solution of 5,7-dichloro-6-(2,6-difluoro-4-iodophenyl)-[1,2,4]triazolo[1,5-a]pyrimidine (0.156 g, 0.365 mmol, 1 equiv) in DMF (1 mL) was added (R)-3-methylbutan-2-amine (0.064 mL, 0.048 g, 0.548 mmol, 1.5 equiv) and triethylamine (0.153 mL, 0.111 g, 1.100 mmol, 3 equiv) and the mixture was stirred for 1 hour at rt. The reaction was quenched by addition of water and the aqueous layer was extracted twice with EtOAc. The combined organic fractions were washed with brine, dried over MgSO₄ and concentrated under reduced pressure. Purification via silica gel column chromatography (Hexanes/EtOAc; 100/0 to 70/30) to furnish the title compound as an off-white solid (0.161 g, 0.337 mmol, 92%). ¹H NMR (600 MHz, CDCl₃) δ 8.32 (s, 1H), 7.46 (d, J=6.5 Hz, 2H), 6.35 (d, J=7.8 Hz, 1H), 3.18 (s, 1H), 1.64 (dq, J=13.2, 6.7 Hz, 1H), 1.06 (d, J=6.6 Hz, 3H), 0.79 (dd, J=11.6, 6.8 Hz, 6H) ppm. ¹³C NMR (151 MHz, CDCl₃) δ 159.60 (dd, J=254.9, 8.4 Hz), 159.56 (dd, J=254.8, 8.4 Hz), 156.51, 153.82, 152.51, 144.76, 120.65 (dt, J=24.4, 3.7 Hz), 110.04 (t, J=21.1 Hz), 93.34 (t, J=10.2 Hz), 87.81, 53.81, 32.50, 16.95, 16.94, 16.77 ppm. HRMS (ES+) calculated for C₁₆H₁₆N₅F₂ICl [M+H]⁺: 478.0101, found 478.0098.

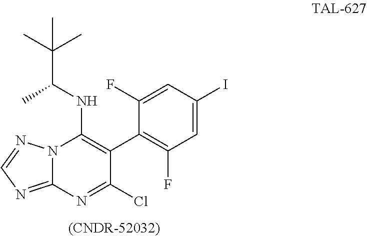

(R)-5-chloro-6-(2,6-difluoro-4-iodophenyl)-N-(3,3-dimethylbutan-2-yl)-[1,2,4]triazolo[1,5-a]pyrimidin-7-amine. To a solution of 5,7-dichloro-6-(2,6-difluoro-4-iodophenyl)-[1,2,4]triazolo[1,5-a]pyrimidine (0.156 g, 0.365 mmol, 1 equiv) in DMF (1 mL) was added (R)-3,3-dimethylbutan-2-amine hydrochloride (0.075 g, 0.548 mmol, 1.5 equiv) and triethylamine (0.153 mL, 0.111 g, 1.100 mmol, 3 equiv) and the mixture was stirred for 1 hour at rt. The reaction was quenched by addition of water and the aqueous layer was extracted twice with EtOAc. The combined organic fractions were washed with brine, dried over MgSO₄ and concentrated under reduced pressure. Purification via silica gel column chromatography (Hexanes/EtOAc; 100/0 to 70/30) to furnish the title compound as an off-white solid (0.158 g, 0.321 mmol, 88%). ¹H NMR (600 MHz, CDCl₃) δ 8.31 (s, 1H), 7.46 (d, J=6.4 Hz, 2H), 6.42 (s, 1H), 3.10 (s, 1H), 1.00 (d, J=6.7 Hz, 4H), 0.82 (s, 9H) ppm. ¹³C NMR (151 MHz, CDCl₃) δ 160.67 (dd, J=254.9, 6.2 Hz), 160.40 (dd, J=254.8, 6.0 Hz), 157.59, 154.89, 153.58, 146.03, 121.82 (dd, J=21.7, 3.9 Hz), 121.67 (dd, J=21.7, 3.9 Hz), 111.23 (t, J=18.2 Hz), 94.41 (t, J=10.1 Hz), 88.71, 58.10, 34.74, 25.77, 16.56, 16.55 ppm. HRMS (ES+) calculated for C₁₇H₁₈N₅F₂ICl [M+H]⁺: 492.0258, found 492.0256.

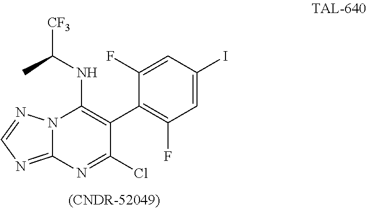

(S)-5-chloro-6-(2,6-difluoro-4-iodophenyl)-N-(1,1,1-trifluoropropan-2-yl)-[1,2,4]triazolo[1,5-a]pyrimidin-7-amine. To a solution of 5,7-dichloro-6-(2,6-difluoro-4-iodophenyl)-[1,2,4]triazolo[1,5-a]pyrimidine (0.200 g, 0.468 mmol, 1 equiv) in DMF (2 mL) was added (S)-1,1,1-trifluoropropan-2-amine (0.097 mL, 0.111 g, 0.984 mmol, 2.1 equiv) and the mixture was stirred for 24 hours at 40° C. The reaction was quenched by addition of water and the aqueous layer was extracted twice with EtOAc. The combined organic fractions were washed with brine, dried over MgSO₄ and concentrated under reduced pressure. Purification via silica gel column chromatography (Hexanes/EtOAc: 100/0 to 70/30) to furnish the title compound as an off-white solid (0.121 g, 0.240 mmol, 51%). ¹H NMR (600 MHz, CDCl₃) δ 8.38 (s, 1H), 7.49 (d, J=6.6 Hz, 2H), 5.97 (d, J=9.6 Hz, 1H), 4.84 (bs, 1H), 1.43 (d, J=6.8 Hz, 3H). ¹³C NMR (151 MHz, CDCl₃) δ 160.61 (dd, J=257.4, 5.9 Hz), 160.29 (dd, J=255.8, 6.0 Hz), 157.41, 155.41, 154.26, 145.87, 124.59 (q, J=282.1 Hz), 122.32 (dd, J=63.0, 3.8 Hz), 122.16 (dd, J=62.9, 3.7 Hz), 109.10 (t, J=20.2 Hz), 95.55 (t, J=10.1 Hz), 91.74, 51.08 (q, J=32.1 Hz), 15.16 ppm. HRMS (ES+) calculated for C₁₄H₉N₅F₅ICl [M+H]⁺: 503.9506, found 503.9502.

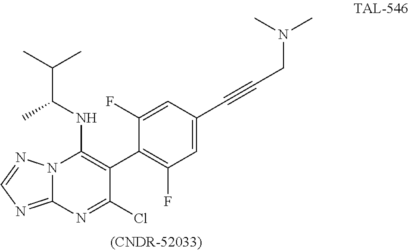

(R)-5-chloro-6-(4-(3-(dimethylamino)prop-1-yn-1-yl)-2,6-difluorophenyl)-N-(3-methylbutan-2-yl)-[1,2,4]triazolo[1,5-a]pyrimidin-7-amine. Following General procedure A using TAL-626 (0.070 g, 0.147 mmol) and N,N-dimethylprop-2-yn-1-amine (0.047 mL, 0.037 g, 0.440 mmol) furnish the title compound as an off-white after purification by reverse phase HPLC and extraction with EtOAc of the pure fractions (0.043 g, 0.099 mmol, 68%). ¹H NMR (600 MHz, MeOD) δ 8.60 (s, 1H), 7.33 (d, J=8.4 Hz, 2H), 3.74 (s, 2H), 3.38 (s, 1H), 2.56 (s, 6H), 1.72 (pt, J=6.4 Hz, 1H), 1.11 (d, J=6.3 Hz, 3H), 0.79 (d, J=6.5 Hz, 6H) ppm. HRMS (ES+) calculated for C₂₁H₂₄N₆F₂Cl [M+H]⁺: 433.1714, found 433.1715.

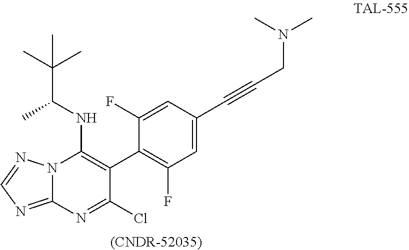

(R)-5-chloro-6-(4-(3-(dimethylamino)prop-1-yn-1-yl)-2,6-difluorophenyl)-N-(3,3-dimethylbutan-2-yl)-[1,2,4]triazolo[1,5-a]pyrimidin-7-amine. Following General procedure A using TAL-627 (0.070 g, 0.142 mmol) and N,N-dimethylprop-2-yn-1-amine (0.046 mL, 0.036 g, 0.427 mmol) furnish the title compound as an off-white after purification by reverse phase HPLC and extraction with EtOAc of the pure fractions (0.043 g, 0.096 mmol, 68%). ¹H NMR (600 MHz, MeOD) δ 8.50 (s, 1H), 7.33 (d, J=7.3 Hz, 2H), 3.58 (bs, 2H), 3.31 (bs, 1H), 2.42 (s, 6H), 1.08 (d, J=6.6 Hz, 3H), 0.83 (s, 9H) ppm. HRMS (ES+) calculated for C₂₂H₂₆N₆F₂Cl [M+H]⁺: 447.1870, found 447.1872.

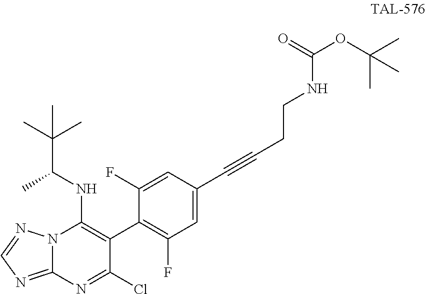 [SMILES: C[C@@H](Nc1c(-c2c(F)cc(C#CCCNC(=O)OC(C)(C)C)cc2F)c(Cl)nc2ncnn12)C(C)(C)C]

tert-butyl(R)-(4-(4-(5-chloro-7-((3-methylbutan-2-yl)amino)-[1,2,4]triazolo[1,5-a]pyrimidin-6-yl)-3,5-difluorophenyl)but-3-yn-1-yl)carbamate. Following general procedure A, using TAL-626 (0.040 g, 0.084 mmol) and tert-butyl but-3-yn-1-ylcarbamate (0.043 g, 0.250 mmol) furnish the title compound after purification by silica gel chromatography (Hexanes/EtOAc: 90/10 to 60/40) as a yellow solid (0.043 g, 0.083 mmol, 99%). ¹H NMR (600 MHz, CDCl₃) δ 8.34 (s, 1H), 7.10 (d, J=7.9 Hz, 2H), 6.34 (s, 1H), 4.85 (s, 1H), 3.40 (t, J=5.9 Hz, 2H), 3.19 (s, 1H), 2.66 (t, J=6.5 Hz, 2H), 1.66-1.62 (m, 1H), 1.47 (s, 9H), 1.06 (d, J=6.6 Hz, 3H), 0.80 (t, J=7.4 Hz, 6H) ppm. HRMS (ES+) calculated for C₂₅H₂₉N₆F₂ClO₂ [M+Na]⁺: 519.2081, found 519.2083.

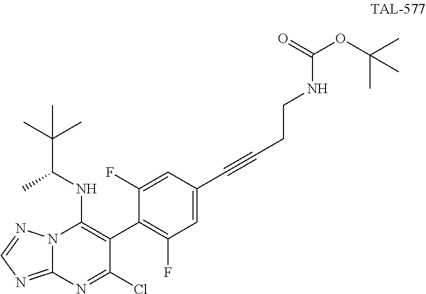 [SMILES: C[C@@H](Nc1c(-c2c(F)cc(C#CCCNC(=O)OC(C)(C)C)cc2F)c(Cl)nc2ncnn12)C(C)(C)C]

tert-butyl(R)-(4-(4-(5-chloro-7-((3,3-dimethylbutan-2-yl)amino)-[1,2,4]triazolo[1,5-a]pyrimidin-6-yl)-3,5-difluorophenyl)but-3-yn-1-yl)carbamate. Following general procedure A, using TAL-627 (0.040 g, 0.081 mmol) and tert-butyl but-3-yn-1-ylcarbamate (0.041 g, 0.240 mmol) furnish the title compound after purification by silica gel chromatography (Hexanes/EtOAc: 90/10 to 60/40) as a yellow solid (0.042 g, 0.079 mmol, 97%). ¹H NMR (600 MHz, CDCl₃) δ 8.40 (s, 1H), 7.11 (d, J=7.7 Hz, 2H), 6.43 (bs, 1H), 4.85 (bs, 1H), 3.41 (d, J=5.1 Hz, 2H), 3.14 (bs, 1H), 2.66 (t, J=6.4 Hz, 2H), 1.47 (s, 9H), 1.02 (d, J=6.6 Hz, 3H), 0.84 (s, 9H) ppm. HRMS (ES+) calculated for C₂₆H₃₂N₆F₂ClO₂ [M+H]⁺: 533.2238, found 533.2238.

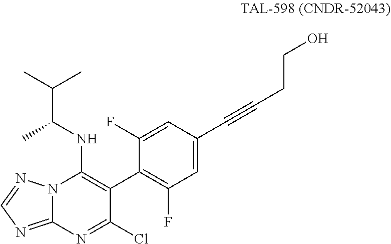 [SMILES: CC(C)[C@@H](C)Nc1c(-c2c(F)cc(C#CCCO)cc2F)c(Cl)nc2ncnn12]

(R)-4-(4-(5-chloro-7-((3-methylbutan-2-yl)amino)-[1,2,4]triazolo[1,5-a]pyrimidin-6-yl)-3,5-difluorophenyl)but-3-yn-1-ol. Following General procedure A using TAL-626 (0.040 g, 0.084 mmol) and but-3-yn-1-ol (0.019 mL, 0.018 g, 0.250 mmol) furnish the title compound as an off-white after purification by reverse phase HPLC and lyophilization (0.012 g, 0.029 mmol, 34%). ¹H NMR (600 MHz, MeOD) δ 8.44 (s, 1H), 7.26 (d, J=8.4 Hz, 2H), 3.77 (t, J=6.5 Hz, 2H), 3.31 (bs, 1H), 2.68 (t, J=6.5 Hz, 2H), 1.80-1.66 (m, 1H), 1.11 (d, J=6.6 Hz, 3H), 0.80 (d, J=6.7 Hz, 6H) ppm. HRMS (ES+) calculated for C₂₀H₂₁N₅F₂ClO [M+H]⁺: 420.1397, found 420.1398.

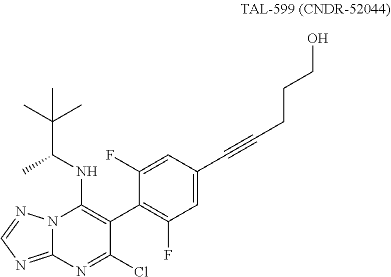 [SMILES: C[C@@H](Nc1c(-c2c(F)cc(C#CCCCO)cc2F)c(Cl)nc2ncnn12)C(C)(C)C]

(R)-5-(4-(5-chloro-7-((3-methylbutan-2-yl)amino)-[1,2,4]triazolo[1,5-a]pyrimidin-6-yl)-3,5-difluorophenyl)pent-4-yn-1-ol. Following General procedure A using TAL-626 (0.027 g, 0.057 mmol) and pent-4-yn-1-ol (0.016 mL, 0.014 g, 0.170 mmol) furnish the title compound as an off-white after purification by reverse phase HPLC and lyophilization (0.012 g, 0.028 mmol, 49%).

¹H NMR (600 MHz, MeOD) δ 8.43 (s, 1H), 7.22 (d, J=8.4 Hz, 2H), 3.71 (t, J=6.2 Hz, 2H), 3.31 (bs, 1H), 2.58 (t, J=7.1 Hz, 2H), 1.84 (p, J=6.7 Hz, 2H), 1.73 (dq, J=13.6, 6.8 Hz, 2H), 1.11 (d, J=6.6 Hz, 3H), 0.80 (d, J=6.7 Hz, 6H) ppm. HRMS (ES+) calculated for C₂₁H₂₃N₅F₂ClO [M+H]⁺: 434.1554, found 434.1553.

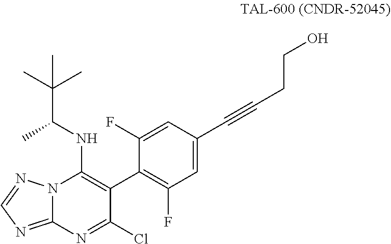 [SMILES: C[C@@H](Nc1c(-c2c(F)cc(C#CCCO)cc2F)c(Cl)nc2ncnn12)C(C)(C)C]

(R)-4-(4-(5-chloro-7-((3,3-dimethylbutan-2-yl)amino)-[1,2,4]triazolo[1,5-a]pyrimidin-6-yl)-3,5-difluorophenyl)but-3-yn-1-ol. Following General procedure A using TAL-627 (0.040 g, 0.081 mmol) and but-3-yn-1-ol (0.018 mL, 0.017 g, 0.240 mmol) furnish the title compound as an off-white after purification by reverse phase HPLC and lyophilization (0.014 g, 0.032 mmol, 40%). ¹H NMR (600 MHz, MeOD) δ 8.46 (s, 1H), 7.28 (dd, J=8.1, 1.5 Hz, 2H), 3.77 (t, J=6.5 Hz, 2H), 3.31 (bs, 1H), 2.68 (t, J=6.5 Hz, 2H), 1.07 (d, J=6.7 Hz, 3H), 0.84 (s, 9H) ppm. HRMS (ES+) calculated for C₂₁H₂₃N₅F₂ClO [M+H]⁺: 434.1554, found 434.1556.

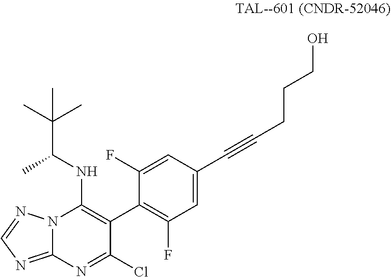 [SMILES: C[C@@H](Nc1c(-c2c(F)cc(C#CCCCO)cc2F)c(Cl)nc2ncnn12)C(C)(C)C]

(R)-5-(4-(5-chloro-7-((3,3-dimethylbutan-2-yl)amino)-[1,2,4]triazolo[1,5-a]pyrimidin-6-yl)-3,5-difluorophenyl)pent-4-yn-1-ol. Following General procedure A using TAL-627 (0.040 g, 0.081 mmol) and pent-4-yn-1-ol (0.023 mL, 0.021 g, 0.240 mmol) furnish the title compound as an off-white after purification by reverse phase HPLC and lyophilization (0.036 g, 0.031 mmol, 38%). ¹H NMR (600 MHz, MeOD) δ 8.46 (s, 1H), 7.25 (dd, J=8.0, 1.5 Hz, 2H), 3.72 (t, J=6.2 Hz, 2H), 3.31 (bs, 1H), 2.58 (t, J=7.1 Hz, 2H), 1.85 (p, J=6.7 Hz, 2H), 1.08 (d, J=6.7 Hz, 3H), 0.84 (s, 9H) ppm. HRMS (ES+) calculated for C₂₂H₂₅N₅F₂ClO [M+H]⁺: 448.1710, found 448.1711.

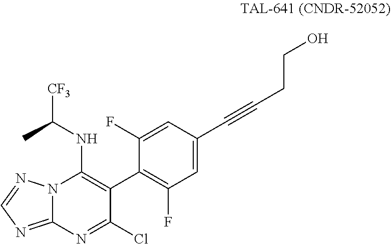 [SMILES: C[C@H](Nc1c(-c2c(F)cc(C#CCCO)cc2F)c(Cl)nc2ncnn12)C(F)(F)F]

(S)-4-(4-(5-chloro-7-((1,1,1-trifluoropropan-2-yl)amino)-[1,2,4]triazolo[1,5-a]pyrimidin-6-yl)-3,5-difluorophenyl)but-3-yn-1-ol. Following General procedure A using TAL-628 (0.032 g, 0.064 mmol) and but-3-yn-1-ol (0.014 mL, 0.013 g, 0.190 mmol) furnish the title compound as an off-white after purification by reverse phase HPLC and lyophilization (0.011 g, 0.025 mmol, 39%). ¹H NMR (600 MHz, MeOD) δ 8.50 (s, 1H), 7.24 (d, J=9.0 Hz, 2H), 5.77 (bs, 1H), 3.76 (t, J=5.9 Hz, 2H), 3.32 (bs, 1H), 2.68 (t, J=5.9 Hz, 2H), 1.46 (d, J=6.5 Hz, 3H) ppm. HRMS (ES+) calculated for C₁₈H₁₄N₅F₅ClO [M+H]⁺: 446.0802, found 446.0804.

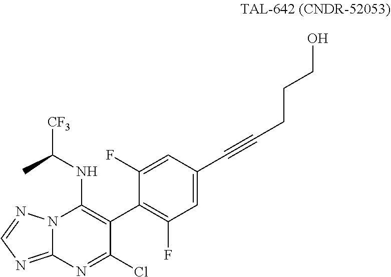 [SMILES: C[C@H](Nc1c(-c2c(F)cc(C#CCCCO)cc2F)c(Cl)nc2ncnn12)C(F)(F)F]

(S)-5-(4-(5-chloro-7-((1,1,1-trifluoropropan-2-yl)amino)-[1,2,4]triazolo[1,5-a]pyrimidin-6-yl)-3,5-difluorophenyl)pent-4-yn-1-ol. Following General procedure A using TAL-628 (0.033 g, 0.066 mmol) and pent-4-yn-1-ol (0.020 mL, 0.017 g, 0.200 mmol) furnish the title compound as an off-white after purification by reverse phase HPLC and lyophilization (0.010 g, 0.022 mmol, 33%). ¹H NMR (600 MHz, MeOD) δ 8.50 (s, 1H), 7.21 (d, J=8.8 Hz, 2H), 5.80 (bs, 1H), 3.71 (t, J=6.0 Hz, 2H), 3.32 (bs, 1H), 2.57 (t, J=7.0 Hz, 2H), 1.94-1.71 (m, 2H), 1.46 (d, J=6.7 Hz, 3H) ppm. HRMS (ES+) calculated for C₁₉H₁₆N₅F₅ClO [M+H]⁺: 460.0958, found 460.0961.

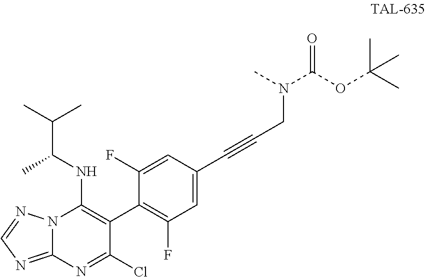

tert-butyl (R)-(3-(4-(5-chloro-7-((3-methylbutan-2-yl)amino)-[1,2,4]triazolo[1,5-a]pyrimidin-6-yl)-3,5-difluorophenyl)prop-2-yn-1-yl)(methyl)carbamate. Following general procedure A, using TAL-626 (0.050 g, 0.105 mmol) and tert-butyl methyl(prop-2-yn-1-yl)carbamate (0.053 g, 0.314 mmol) furnish the title compound after purification by silica gel chromatography (Hexanes/EtOAc: 90/10 to 50/50) as a yellow solid (0.020 g, 0.039 mmol, 37%). ¹H NMR (600 MHz, CDCl₃) δ 8.33 (s, 1H), 7.12 (d, J=8.0 Hz, 2H), 6.35 (d, J=8.1 Hz, 1H), 4.32 (bs, 2H), 3.16 (bs, 1H), 2.99 (s, 3H), 1.65-1.60 (m, 1H), 1.49 (s, 9H), 1.05 (d, J=6.6 Hz, 3H), 0.80 (d, J=6.9 Hz, 3H), 0.78 (d, J=6.9 Hz, 3H) ppm. HRMS (ES+) calculated for C₂₅H₃₀N₆F₂ClO₂ [M+H]⁺: 519.2081, found 519.2078.

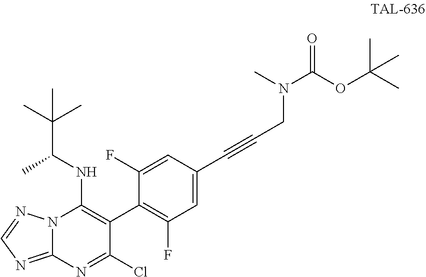 [SMILES: C[C@@H](Nc1c(-c2c(F)cc(C#CCN(C)C(=O)OC(C)(C)C)cc2F)c(Cl)nc2ncnn12)C(C)(C)C]

tert-butyl (R)-(3-(4-(5-chloro-7-((3,3-dimethylbutan-2-yl)amino)-[1,2,4]triazolo[1,5-a]pyrimidin-6-yl)-3,5-difluorophenyl)prop-2-yn-1-yl)(methyl)carbamate. Following general procedure A, using TAL-627 (0.050 g, 0.102 mmol) and tert-butyl methyl(prop-2-yn-1-yl)carbamate (0.052 g, 0.305 mmol) furnish the title compound after purification by silica gel chromatography (Hexanes/EtOAc: 90/10 to 50/50) as a yellow solid (0.043 g, 0.081 mmol, 79%). ¹H NMR (600 MHz, CDCl₃) δ 8.32 (s, 1H), 7.12 (d, J=7.5 Hz, 2H), 6.43 (bs, 1H), 4.31 (bs, 2H), 2.98 (s, 3H), 1.49 (s, 9H), 1.47-1.45 (m, 1H), 1.01 (d, J=6.7 Hz, 3H), 0.83 (s, 9H) ppm. HRMS (ES+) calculated for C₂₆H₃₂N₆F₂ClO₂ [M+H]⁺: 533.2238, found 533.2234.

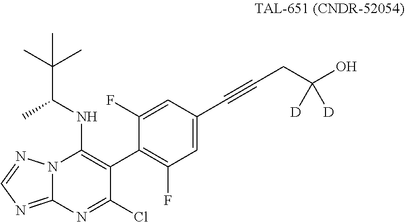 [SMILES: [2H]C([2H])(O)CC#Cc1cc(F)c(-c2c(Cl)nc3ncnn3c2N[C@H](C)C(C)(C)C)c(F)c1]

(R)-4-(4-(5-chloro-7-((3,3-dimethylbutan-2-yl)amino)-[1,2,4]triazolo[1,5-a]pyrimidin-6-yl)-3,5-difluorophenyl)but-3-yn-1,1-d₂-1-ol. Following General procedure A using TAL-627 (0.080 g, 0.160 mmol) and but-3-yn-1,1-d₂-1-ol (0.035 g, 0.490 mmol) furnish the title compound as an off-white after purification by reverse phase HPLC and lyophilization (0.031 g, 0.071 mmol, 44%). ¹H NMR (600 MHz, MeOD) δ 8.46 (s, 1H), 7.27 (d, J=7.7 Hz, 2H), 3.32 (bs, 1H), 2.67 (s, 2H), 1.06 (d, J=6.7 Hz, 3H), 0.82 (s, 9H) ppm. HRMS (ES+) calculated for C₂₁H₂₁N₅F₂D₂ClO [M+H]⁺: 436.1679, found 436.1678.

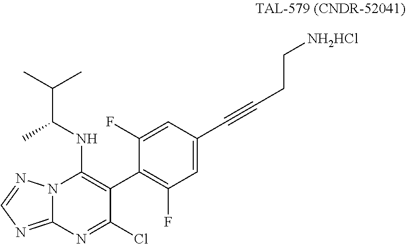

(R)-6-(4-(4-aminobut-1-yn-1-yl)-2,6-difluorophenyl)-5-chloro-N-(3-methylbutan-2-yl)-[1,2,4]triazolo[1,5-a]pyrimidin-7-amine hydrochloride. Following general procedure B, TAL-576 (0.040 g, 0.077 mmol), was converted to the title compound (0.034 g, 0.075 mmol, 97%) as a yellow solid. ¹H NMR (600 MHz, MeOD) δ 8.87 (s, 1H), 7.37 (d, J=8.3 Hz, 2H), 3.33 (bs, 1H), 3.23 (t, J=6.7 Hz, 2H), 2.92 (t, J=6.8 Hz, 2H), 1.77 (dq, J=13.6, 6.8 Hz, 1H), 1.15 (d, J=6.6 Hz, 3H), 0.81 (t, J=7.3 Hz, 6H) ppm. HRMS (ES+) calculated for C₂₀H₂₂N₆F₂Cl [M+H]⁺: 419.1557, found 419.1560.

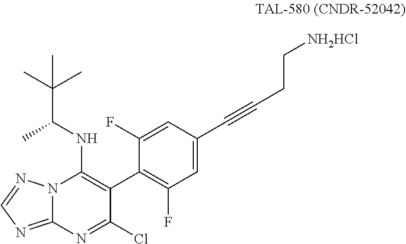

(R)-6-(4-(4-aminobut-1-yn-1-yl)-2,6-difluorophenyl)-5-chloro-N-(3,3-dimethylbutan-2-yl)-[1,2,4]triazolo[1,5-a]pyrimidin-7-amine hydrochloride. Following general procedure B, TAL-576 (0.040 g, 0.075 mmol), was converted to the title compound (0.034 g, 0.072 mmol, 97%) as a yellow solid. ¹H NMR (600 MHz, MeOD) δ 8.75 (s, 1H), 7.38 (dd, J=8.8, 3.6 Hz, 2H), 3.33 (bs, 1H), 3.23 (t, J=6.8 Hz, 2H), 2.92 (t, J=6.8 Hz, 2H), 1.34-1.30 (m, 1H), 1.10 (d, J=6.7 Hz, 3H), 0.84 (s, 9H) ppm. HRMS (ES+) calculated for C₂₁H₂₄N₆F₂Cl [M+H]⁺: 433.1714, found 433.1716.

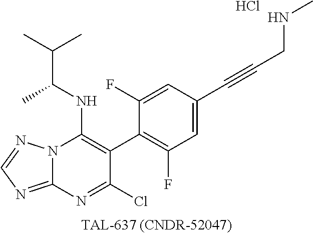 [SMILES: CNCC#Cc1cc(F)c(-c2c(Cl)nc3ncnn3c2N[C@H](C)C(C)C)c(F)c1 ; Cl]

(R)-5-chloro-6-(2,6-difluoro-4-(3-(methylamino)prop-1-yn-1-yl)phenyl)-N-(3-methylbutan-2-yl)-[1,2,4]triazolo[1,5-a]pyrimidin-7-amine hydrochloride. Following general procedure B, TAL-635 (0.015 g, 0.029 mmol), was converted to the title compound (0.011 g, 0.024 mmol, 84%) as a brown solid. ¹H NMR (600 MHz, MeOD) δ 8.59 (s, 1H), 7.42 (d, J=8.2 Hz, 2H), 4.23 (s, 2H), 3.31 (bs, 1H), 2.85 (s, 3H), 1.74 (dq, J=13.4, 6.7 Hz, 1H), 1.11 (d, J=6.5 Hz, 3H), 0.79 (d, J=4.4 Hz, 6H) ppm. HRMS (ES+) calculated for C₂₀H₂₂N₆F₂Cl [M+H]⁺: 419.1557, found 419.1554.

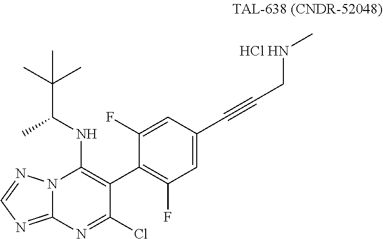

(R)-5-chloro-6-(2,6-difluoro-4-(3-(methylamino)prop-1-yn-1-yl)phenyl)-N-(3,3-dimethylbutan-2-yl)-[1,2,4]triazolo[1,5-a]pyrimidin-7-amine hydrochloride. Following general procedure B, TAL-636 (0.035 g, 0.066 mmol), was converted to the title compound (0.028 g, 0.060 mmol, 91%) as a brown solid. ¹H NMR (600 MHz, MeOD) δ 8.74 (s, 1H), 7.47 (dd, J=8.4, 3.1 Hz, 2H), 4.24 (s, 2H), 3.33 (bs, 1H), 2.86 (s, 3H), 1.10 (d, J=6.7 Hz, 3H), 0.84 (s, 9H) ppm. HRMS (ES+) calculated for C₂₁H₂₄N₆F₂Cl [M+H]⁺: 433.1714, found 433.1716.

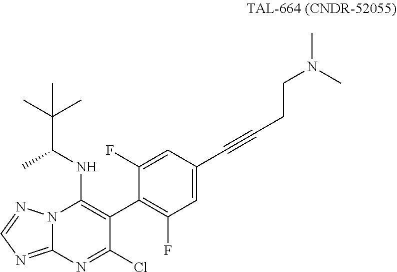 [SMILES: C[C@@H](Nc1c(-c2c(F)cc(C#CCCN(C)C)cc2F)c(Cl)nc2ncnn12)C(C)(C)C]

(R)-5-chloro-6-(4-(4-(dimethylamino)but-1-yn-1-yl)-2,6-difluorophenyl)-N-(3,3-dimethylbutan-2-yl)-[1,2,4]triazolo[1,5-a]pyrimidin-7-amine. Following General procedure A using TAL-627 (0.040 g, 0.081 mmol) and N,N-dimethylbut-3-yn-1-amine (0.029 mL, 0.024 g, 0.224 mmol) furnish the title compound as an off-white after purification by reverse phase HPLC and extraction with EtOAc (0.023 g, 0.050 mmol, 61%). ¹H NMR (600 MHz, CDCl₃) δ 8.36 (s, 1H), 7.13 (d, J=6.8 Hz, 2H), 6.44 (bs, 1H), 3.34 (s, 2H), 3.23-3.01 (m, 3H), 2.93 (s, 6H), 1.01 (d, J=6.3 Hz, 3H), 0.82 (s, 9H) ppm.

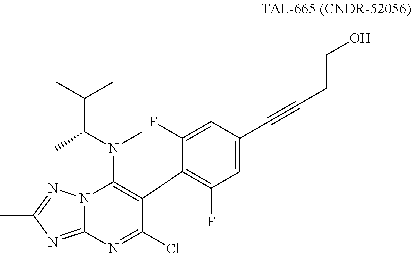 [SMILES: Cc1nc2nc(Cl)c(-c3c(F)cc(C#CCCO)cc3F)c(N(C)[C@H](C)C(C)C)n2n1]

(R)-4-(4-(5-chloro-2-methyl-7-(methyl(3-methylbutan-2-yl)amino)-[1,2,4]triazolo[1,5-a]pyrimidin-6-yl)-3,5-difluorophenyl)but-3-yn-1-ol. To a solution of TAL-626 (0.037 g, 0.077 mmol, 1 equiv) in DMF (0.8 mL) at 0° C. was added NaH (60%, 0.005 g, 0.120 mmol, 1.5 equiv) followed by methyl iodide after 20 min (0.006 mL, 0.013 g, 0.093 mmol, 1.2 equiv). After 2 hours at rt the reaction was quenched by addition of water and the aqueous layer was extracted with EtOAc twice. The combined organic fractions were washed with brine, dried over MgSO₄ and concentrated under reduced pressure to furnish the bis-methylated intermediate which was used in the next step without further purification. Following General procedure A using the previous intermediate (0.013 g, 0.026 mmol) and but-3-yn-1-ol (0.006 mL, 0.005 g, 0.077 mmol) furnish the title compound as an off-white after purification by reverse phase HPLC and extraction with EtOAc (0.006 g, 0.013 mmol, 17% over two steps). ¹H NMR (600 MHz, CDCl₃) δ 7.02 (d, J=7.1 Hz, 1H), 4.04 (bs, 1H), 3.84 (s, 2H), 3.42 (s, 3H), 2.71 (s, 2H), 2.54 (s, 3H), 1.65-1.60 (m, 1H), 1.05 (d, J=5.9 Hz, 3H), 0.85 (d, J=5.5 Hz, 3H), 0.76 (d, J=5.8 Hz, 3H) ppm.

1. A compound of formula I: 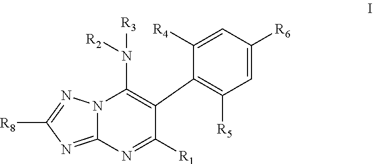
wherein
R₁ is Cl;
R₂ is CH(CH₃)CF₃, CH(isopropyl)CF₃, CH(isopropyl)CH₃, CH(tert-butyl)CH₃, or CH(methyl diazirinyl)CH₃, wherein R₂ may optionally be mono- or poly-deuterated;
R₃ is H or alkyl; or, R₂ and R₃, together with the N atom to which they are attached, form a C₅-C₇ heterocyclic ring or 3-methoxy-8-azabicyclo[3.2.1]octan-8-yl;
R₄ is H or F;
R₅ is H or F;
R₆ is iodo, cyano, ethynyl, or —C≡C—(CH₂)ₙ—R₇, wherein n=0-3, and R₇ is H, —OH, —NH₂, —NHCH₃, or —N(CH₃)₂, wherein R₆ may optionally be mono- or poly-deuterated;
R₅ is H or alkyl;
or a stereochemical isomer thereof;
or a pharmaceutically acceptable salt thereof.

2. The compound according to claim 1, wherein R₆ is cyano.

3. The compound according to claim 1, wherein R₄ and R₅ are both F.

4. The compound according to claim 3, wherein R₂ is CH(CH₃)CF₃, CH(isopropyl)CF₃, CH(isopropyl)CH₃, or CH(tert-butyl)CH₃.

5. The compound according to claim 3, wherein R₂ and R₃, together with the N atom to which they are attached, form piperidinyl, azepanyl, or azocanyl.

6. The compound according to claim 1, wherein R₄ is H and R₅ is F.

7. The compound according to claim 6, wherein R₂ is CH(CH₃)CF₃, CH(isopropyl)CF₃, CH(isopropyl)CH₃, or CH(tert-butyl)CH₃.

8. The compound according to claim 6, wherein R₂ and R₃, together with the N atom to which they are attached, form piperidinyl, azepanyl, or azocanyl.

9. The compound according to claim 1, wherein R₆ is ethynyl.

10. The compound according to claim 9, wherein R₄ and R₅ are both F.

11. The compound according to claim 10, wherein R₂ is CH(CH₃)CF₃, CH(isopropyl)CF₃, CH(isopropyl)CH₃, or CH(tert-butyl)CH₃.

12. The compound according to claim 10, wherein R₂ and R₃, together with the N atom to which they are attached, form piperidinyl, azepanyl, or azocanyl.

13. The compound according to claim 9, wherein R₄ is H and R₅ is F.

14. The compound according to claim 13, wherein R₂ is CH(CH₃)CF₃, CH(isopropyl)CF₃, CH(isopropyl)CH₃, or CH(tert-butyl)CH₃.

15. The compound according to claim 13, wherein R₂ and R₃, together with the N atom to which they are attached, form piperidinyl, azepanyl, or azocanyl.

16. The compound according to claim 1, wherein the compound is

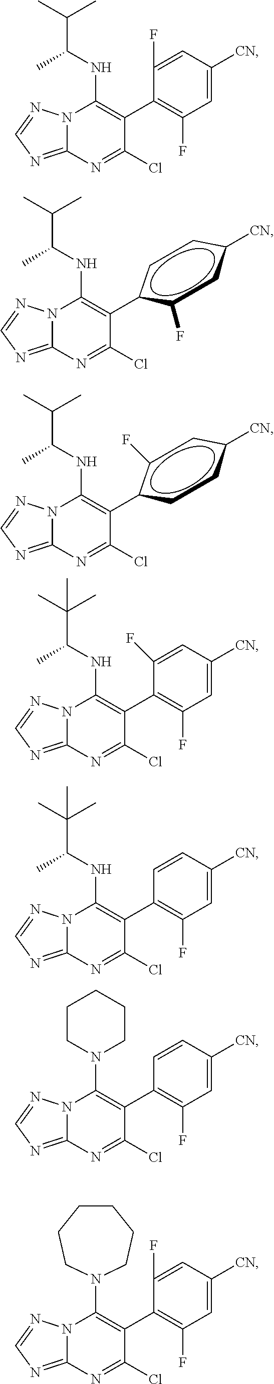

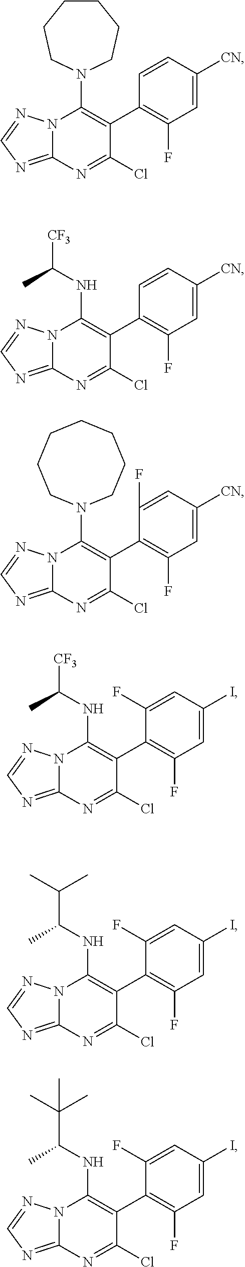

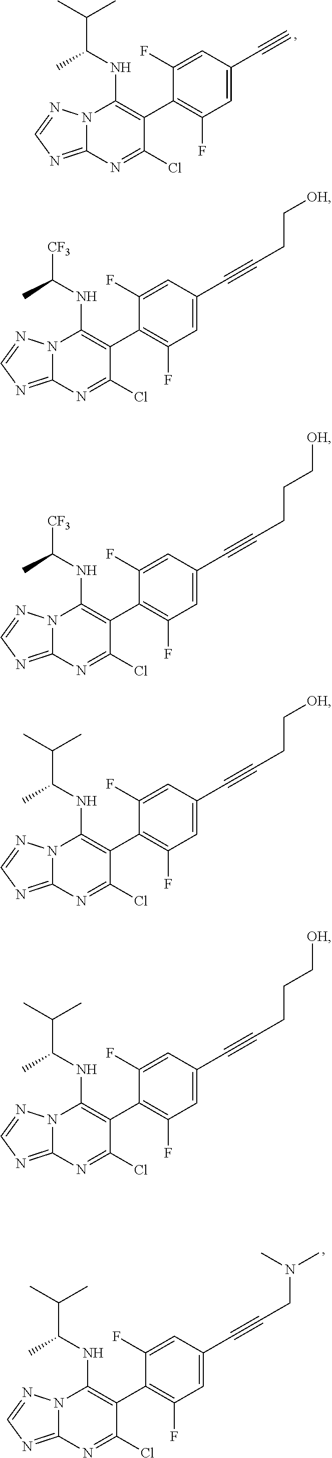 [SMILES: C#Cc1cc(F)c(-c2c(Cl)nc3ncnn3c2N[C@H](C)C(C)C)c(F)c1 ; C[C@H](Nc1c(-c2c(F)cc(C#CCCO)cc2F)c(Cl)nc2ncnn12)C(F)(F)F ; C[C@H](Nc1c(-c2c(F)cc(C#CCCCO)cc2F)c(Cl)nc2ncnn12)C(F)(F)F ; CC(C)[C@@H](C)Nc1c(-c2c(F)cc(C#CCCO)cc2F)c(Cl)nc2ncnn12 ; CC(C)[C@@H](C)Nc1c(-c2c(F)cc(C#CCCCO)cc2F)c(Cl)nc2ncnn12 ; CC(C)[C@@H](C)Nc1c(-c2c(F)cc(C#CCN(C)C)cc2F)c(Cl)nc2ncnn12]

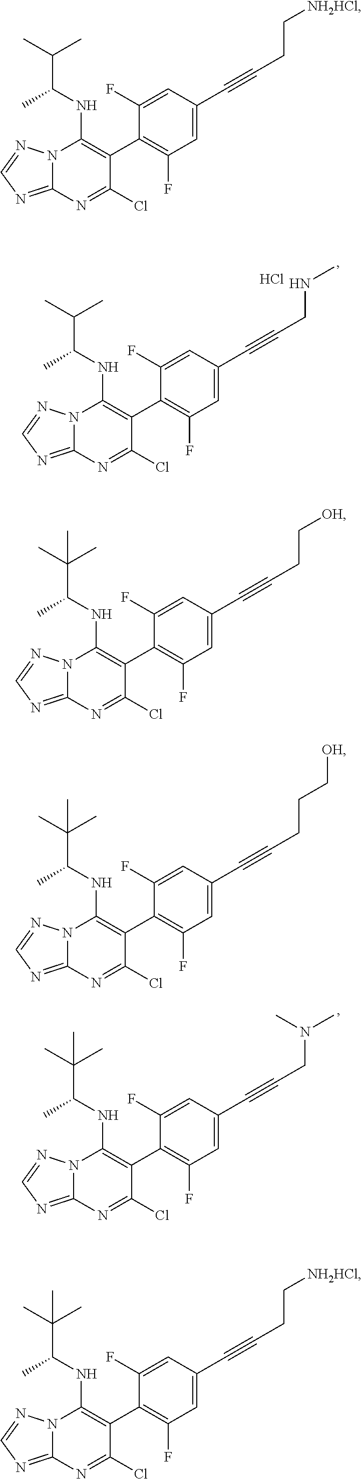

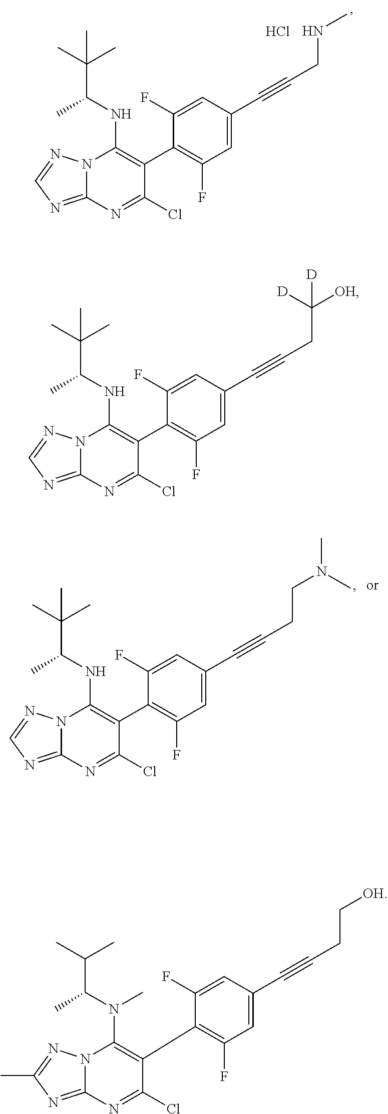

17. A composition for treating a neurodegenerative disease comprising a therapeutically effective amount of a compound according to claim 1.

18. A composition for treating cancer comprising a therapeutically effective amount of a compound according to claim 1.

19. A method of treating a neurodegenerative disease in a subject in need thereof comprising administering to the subject a therapeutically effective amount of a compound according to claim 1.

20. The method according to claim 19, wherein the neurodegenerative disease is characterized by a tauopathy or compromised microtubule function in the brain of the subject.

21. The method according to claim 19, wherein the neurodegenerative disease is Alzheimer's disease, frontotemporal lobar degeneration, Pick's disease, progressive supranuclear palsy (PSP), corticobasal degeneration, Parkinson's disease (PD), PD with dementia, Lewy body disease with dementia, or amyotrophic lateral sclerosis.

22. The method according to claim 19, wherein the neurodegenerative disease is traumatic brain injury or post-traumatic stress disorder.

23. The method according to claim 22, wherein the traumatic brain injury is repetitive traumatic brain injury or chronic traumatic encephalopathy

24. The method according to claim 19, wherein the neurodegenerative disease is schizophrenia.

25. A method of treating cancer in a subject in need thereof comprising administering to the subject a therapeutically effective amount of a compound according to claim 1.

26. The method according to claim 25, wherein the cancer is a brain cancer.

27. The method according to claim 26, wherein the cancer is a glioma or an astrocytoma.
